(19) United States
(12) Patent Application Publication
(10) Pub. No.: US 2024/0101973 A1
(43) Pub. Date: Mar. 28, 2024

(54) METHODS AND COMPOSITIONS FOR PRODUCING VITAMIN K DEPENDENT PROTEINS

(71) Applicant: The University of North Carolina at Chapel Hill, Chapel Hill, NC (US)

(72) Inventors: Darrel W. Stafford, Carrboro, NC (US); Tao Li, San Diego, CA (US)

(21) Appl. No.: 18/184,198

(22) Filed: Mar. 15, 2023

Related U.S. Application Data
(63) Continuation of application No. 17/412,827, filed on Aug. 26, 2021, now abandoned.
Application No. 17/412,827 is a continuation of application No. 16/869,701, filed on May 8, 2020, now abandoned.
Application No. 16/869,701 is a continuation of application No. 16/575,083, filed on Sep. 18, 2019, now abandoned.
Application No. 16/575,083 is a continuation of application No. 16/272,290, filed on Feb. 11, 2019, now abandoned.
Application No. 16/272,290 is a continuation of application No. 16/020,731, filed on Jun. 27, 2018, now abandoned.
Application No. 16/020,731 is a continuation of application No. 15/794,843, filed on Oct. 26, 2017, now abandoned.
Application No. 15/794,843 is a continuation of application No. 15/228,740, filed on Aug. 4, 2016, now abandoned.
Application No. 15/228,740 is a continuation of application No. 14/490,244, filed on Sep. 18, 2014, now Pat. No. 9,441,208.
Application No. 14/490,244 is a continuation of application No. 14/072,108, filed on Nov. 5, 2013, now abandoned.
Application No. 14/072,108 is a continuation of application No. 12/612,154, filed on Nov. 4, 2009, now Pat. No. 8,603,823.
Application No. 12/612,154 is a continuation of application No. 11/787,072, filed on Apr. 13, 2007, now Pat. No. 7,645,602.
Application No. 11/787,072 is a continuation-in-part of application No. 10/573,131, filed on Apr. 18, 2006, now Pat. No. 7,687,233.
Application No. 10/573,131 is a continuation-in-part of application No. PCT/US2005/008643, filed on Mar. 15, 2005.
(60) Provisional application No. 60/505,527, filed on Sep. 23, 2003.

(51) Int. Cl. C12N 9/04; C12N 9/50; C12N 9/64; C12N 9/88; C12Q 1/6883

(52) U.S. Cl. CPC C12N 9/0006; C12N 9/50; C12N 9/64; C12N 9/88; C12Q 1/6883; C12Y 101/04001; C12Q 2600/106; C12Q 2600/156; C12Q 2600/158; C12Q 2600/172; C12Y 304/21006; C12Y 401/0109

(57) ABSTRACT

The present invention relates to methods and compositions for improving the productivity of recombinant vitamin K dependent protein expression in host cells.

### STATEMENT OF PRIORITY

The present application is a continuation application of, and claims priority to, U.S. application Ser. No. 17/412,827, filed Aug. 26, 2021, which is a continuation application of, and claims priority to, U.S. application Ser. No. 16/869,701, filed May 8, 2020 (abandoned), which is a continuation of U.S. application Ser. No. 16/575,083, filed Sep. 18, 2019 (abandoned), which is a continuation application of U.S. application Ser. No. 16/272,290, filed Feb. 11, 2019 (abandoned), which is a continuation application of U.S. application Ser. No. 16/020,731, filed Jun. 27, 2018 (abandoned), which is a continuation application of U.S. application Ser. No. 15/794,843, filed Oct. 26, 2017 (abandoned), which is a continuation application of U.S. application Ser. No. 15/228,740, filed Aug. 4, 2016 (abandoned), which is a continuation application of U.S. application Ser. No. 14/490,244, filed Sep. 18, 2014 and issued as U.S. Pat. No. 9,441,208 on Sep. 13, 2016, which is a continuation application of U.S. application Ser. No. 14/072,108, filed Nov. 5, 2013 (abandoned), which is a continuation application of U.S. application Ser. No. 12/612,154, filed Nov. 4, 2009 and issued as U.S. Pat. No. 8,603,823 on Dec. 12, 2013, which is a continuation application of and claims priority to, U.S. application Ser. No. 11/787,072, filed Apr. 13, 2007, and issued as U.S. Pat. No. 7,645,602 on Jan. 12, 2010, which is a continuation-in-part application of U.S. application Ser. No. 10/573,131, filed Apr. 18, 2006 and issued as U.S. Pat. No. 7,687,233 on Mar. 30, 2010, which claims the benefit, under 35 U.S.C. § 119(e), of U.S. Provisional Application Ser. No. 60/505,527, filed Sep. 23, 2003, and is a continuation-in-part of PCT Application No. PCT/US2005/008643, filed Mar. 15, 2005, the entire contents of each of which are incorporated by reference herein.

### STATEMENT OF GOVERNMENT SUPPORT

This invention was made with government support under Grant Number HL006350 and HL048318 awarded by the National Institutes of Health. The government has certain rights in the invention.

### FIELD OF THE INVENTION

The present invention relates to methods and compositions for improving the productivity of recombinant vitamin K-dependent protein expression in host cells.

### BACKGROUND OF THE INVENTION

The function of numerous proteins requires the modification of multiple glutamic acid residues to γ-carboxyglutamate. Among these vitamin K-dependent (VKD) coagulation proteins, FIX (Christmas factor), FVII, and prothrombin are the best known. The observation that a knock-out of the gene for matrix Gla protein results in calcification of the mouse's arteries (Luo et al. (1997) “Spontaneous calcification of arteries and cartilage in mice lacking matrix GLA protein” Nature 386:78-81) emphasizes the importance of the vitamin K cycle for proteins with functions other than coagulation. Moreover, Gas6 and other Gla proteins of unknown function are expressed in neural tissue and warfarin exposure in utero results in mental retardation and facial abnormalities. This is consistent with the observation that the expression of VKD carboxylase, the enzyme that accomplishes the Gla modification, is temporally regulated in a tissue-specific manner with high expression in the nervous system during early embryonic stages. Concomitant with carboxylation, reduced vitamin K, a co-substrate of the reaction, is converted to vitamin K epoxide. Because the amount of vitamin K in the human diet is limited, vitamin K epoxide must be converted back to vitamin K by vitamin K epoxide reductase (VKOR) to prevent its depletion. Warfarin, the most widely used anticoagulation drug, targets VKOR and prevents the regeneration of vitamin K. The consequence is a decrease in the concentration of reduced vitamin K, which results in a reduced rate of carboxylation by the γ-glutamyl carboxylase and in the production of undercarboxylated vitamin K-dependent proteins.

### SUMMARY OF THE INVENTION

In one embodiment, the present invention relates to an isolated nucleic acid encoding vitamin K epoxide reductase (VKOR), particularly mammalian (e.g., human, ovine, bovine, monkey, etc.) VKOR. Examples include (a) nucleic acids as disclosed herein, such as isolated nucleic acids having the nucleotide sequence as set forth in SEQ ID NO: 8 or SEQ ID NO: 9; (b) nucleic acids that hybridize to isolated nucleic acids of (a) above or the complement thereof (e.g., under stringent conditions), and/or have substantial sequence identity to nucleic acids of (a) above (e.g., are 80, 85, 90 95 or 99% identical to nucleic acids of (a) above), and encode a VKOR; and (c) nucleic acids that differ from the nucleic acids of (a) or (b) above due to the degeneracy of the genetic code, but code for a VKOR encoded by a nucleic acid of (a) or (b) above.

An additional aspect of the present invention is a recombinant nucleic acid comprising a nucleic acid encoding vitamin K epoxide reductase as described herein operatively associated with a heterologous promoter.

A further aspect of the present invention is a cell that contains and expresses a recombinant nucleic acid as described above. Suitable cells include plant, animal, mammal, insect, yeast and bacterial cells.

A further aspect of the present invention is an oligonucleotide that hybridizes to an isolated nucleic acid encoding VKOR as described herein.

A further aspect of the present invention is isolated and purified VKOR (e.g., VKOR purified to homogeneity) encoded by a nucleic acid as described herein. For example, the VKOR of this invention can comprise the amino acid sequence as set forth in SEQ ID NO:10.

A further aspect of the present invention is a method of making a vitamin K dependent protein which comprises culturing a host cell that expresses a nucleic acid encoding a vitamin K dependent protein in the presence of vitamin K and produces a vitamin K dependent protein, and then harvesting the vitamin K dependent protein from the culture, the host cell containing and expressing a heterologous nucleic acid encoding vitamin K dependent carboxylase, and the host cell further containing and expressing a heterologous nucleic acid encoding vitamin K epoxide reductase (VKOR) and producing VKOR as described herein. Thus, several embodiments of the present invention further provide a cell comprising a heterologous nucleic acid encoding vitamin K dependent carboxylase and a heterologous nucleic acid encoding vitamin K epoxide reductase. The cell can further comprise nucleic acid encoding a vitamin K dependent protein, which nucleic acid can be heterologous to the cell or endogenous to the cell.

An isolated and/or purified host cell or organism is disclosed in accordance with an embodiment of the present invention. In one embodiment, the isolated host cell comprises a recombinant nucleic acid coding for a vitamin K epoxide reductase (VKOR) or a functionally active derivative thereof, and a recombinant nucleic acid coding for a vitamin K dependent protein or a functionally active derivative thereof, wherein both the recombinant VKOR and the recombinant Vitamin K dependent protein are expressed in the host cell. One of skill in the art will understand that VKOR and vitamin K reductase complex subunit 1 (VKORC1) refer to the same enzyme, and that the terms can be used interchangeably herein. Although in some embodiments, the host cell can also include vitamin K dependent carboxylase, the carboxylase is an optional compound and need not be included.

In a variation to the isolated host cell, the nucleic acid coding for recombinant VKOR or the nucleic acid coding for the recombinant Vitamin K dependent protein or both are expressed via an expression mode selected from the group consisting of induced, transient, and permanent expression, or combinations thereof.

In another variation, the isolated host cell is a mammalian cell. The mammalian cell may be a cell derived from a mammalian cell line selected from the group consisting of CHO cells and HEK293 cells, or combinations thereof.

In another variation to the isolated host cell, the recombinant Vitamin K dependent protein is a coagulation factor or a functionally active derivative thereof. The coagulation factor may be selected from the group consisting of factor II, factor VII, factor IX, factor X, prothrombin, Protein C and Protein S. In one preferred embodiment, the coagulation factor is human factor IX, or combinations thereof.

A cell culture system is disclosed in accordance with another embodiment of the present invention. The cell culture system comprises isolated cells that contain a recombinant nucleic acid coding for a vitamin K epoxide reductase (VKOR) or a functionally active derivative thereof and a recombinant nucleic acid coding for a vitamin K dependent protein or a functionally active derivative thereof, wherein both the recombinant VKOR and the recombinant Vitamin K dependent protein are expressed in the cells.

In a variation to the cell culture system, the cultured cells are mammalian cells. In another variation, the mammalian cells may be selected from the group consisting of CHO cells and HEK293 cells, or combinations thereof.

In another variation to cell culture system, the recombinant Vitamin K dependent protein is a coagulation factor or a functionally active derivative thereof. Preferably, the coagulation factor is selected from the group consisting of factor II, factor VII, factor IX, factor X, prothrombin, Protein C and Protein S. In one preferred embodiment, the coagulation factor is human factor IX.

A method for improving the productivity of recombinant vitamin K dependent protein expression in an isolated and/or purified host cell or organism is disclosed in accordance with another embodiment of the present invention. In one embodiment, the method comprises the steps of: providing an isolated host cell; inserting a recombinant nucleic acid coding for a Vitamin K dependent protein or a functionally active derivative thereof into the host cell; inserting a recombinant nucleic acid coding for a vitamin K epoxide reductase complex (VKOR) or a functionally active derivative thereof into the host cell; and expressing the recombinant nucleic acids.

Another method for improving the productivity of recombinant vitamin K dependent protein expression in a host cell is disclosed in accordance with another embodiment of the invention. The method comprises the steps of: providing an isolated host cell having a recombinant nucleic acid coding for a Vitamin K dependent protein or a functionally active derivative thereof integrated into its genome; inserting a recombinant nucleic acid coding for a vitamin K epoxide reductase (VKOR) or a functionally active derivative thereof into the host cell; and expressing the nucleic acids. In a variation to the method, the recombinant nucleic acid coding for the Vitamin K dependent protein or a functionally active derivative thereof is stably expressed.

A method is disclosed in accordance with another embodiment of the invention for improving the productivity of recombinant vitamin K dependent protein expression or a functionally active derivative thereof in a host cell. The method comprises the steps of: providing an isolated host cell having a recombinant nucleic acid coding for a vitamin K epoxide reductase (VKOR) or a functionally active derivative thereof integrated into its genome; inserting a recombinant nucleic acid coding for a Vitamin K dependent protein or a functionally active derivative thereof into the host cell; and expressing the nucleic acids. The recombinant nucleic acid coding for VKOR or a functionally active derivative thereof is preferably stably expressed.

A recombinant Vitamin K dependent protein is disclosed in accordance with another embodiment of the invention. The protein is obtainable by inserting a recombinant nucleic acid coding for a Vitamin K epoxide reductase (VKOR) or a functionally active derivative thereof and a recombinant nucleic acid coding for the recombinant Vitamin K dependent protein or a functionally active derivative thereof into a host cell, expressing the nucleic acids, and recovering the recombinant Vitamin K dependent protein.

### BRIEF DESCRIPTION OF THE DRAWINGS

FIGS. 1A-D Comparisons of warfarin dosages in wild type, heterozygous and homozygous subjects for SNPs vk 2581, vk3294 and vk4769, as well as a comparison of warfarin dosages in wild type and heterozygous subjects for P450 2Y9.

FIG. 2. For each of the 13 siRNA pools, three T7 flasks containing A549 cells were transfected and VKOR activity determined after 72 h. The VKOR assay used 25 μM vitamin K epoxide. One siRNA pool specific for gene gi:13124769 reduced VKOR activity by 64%-70% in eight repetitions.

FIG. 3. Time course of inhibition of VKOR activity by the siRNA pool specific for gi:13124769 in A549 cells. VKOR activity decreased continuously during this time period while the level of its mRNA decreased rapidly to about 20% of normal. 25 μM vitamin K epoxide was used for this assay. The siRNA did not affect the activity of VKD carboxylase or the level of lam in NC mRNA.

FIG. 4. VKOR activity was detected when mGC_11276 was expressed in Sf9 insect cells. ˜1×10⁶ cells were used in this assay. Reactions were performed using 32 μM KO at 30° C. for 30 minutes in Buffer D. Blank Sf9 cells served as a negative control and A549 cells as a reference.

FIG. 5. Inhibition of VKOR by warfarin. Reactions were performed using 1.6 mg microsomal proteins made from VKOR_Sf9 cells, 60 μM KO, and various concentration of warfarin at 30° C. for 15 minutes in Buffer D.

FIGS. 6A-D. Carboxylation of a vitamin K dependent protein, factor X. Panel 6A: Control HEK293 cells producing factor X without exogenous VKOR or VKGC. Panel 6B: HEK 293 cells producing factor X and exogenous VKGC alone. Panel 6C: HEK293 cells producing factor X and exogenous VKOR alone. Panel 6D: HEK293 cells producing factor X and both exogenous VKOR and CKGC.

### DETAILED DESCRIPTION OF THE INVENTION

As used herein, “a,” “an” or “the” can mean one or more than one. For example, “a” cell can mean a single cell or a multiplicity of cells.

The present invention is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which do not depart from the instant invention. Hence, the following specification is intended to illustrate some particular embodiments of the invention, and not to exhaustively specify all permutations, combinations and variations thereof.

The “Sequence Listing” attached hereto forms a part of the instant specification as if fully set forth herein.

The present invention may be carried out based on the instant disclosure and further utilizing methods, components and features known in the art, including but not limited to those described in U.S. Pat. No. 5,268,275 to Stafford and Wu and U.S. Pat. No. 6,531,298 to Stafford and Chang, the disclosures of which are incorporated by reference herein in their entirety as if fully set forth herein.

As used herein, “nucleic acids” encompass both RNA and DNA, including cDNA, genomic DNA, synthetic (e.g., chemically synthesized) DNA and chimeras of RNA and DNA. The nucleic acid may be double-stranded or single-stranded. Where single-stranded, the nucleic acid may be a sense strand or an antisense strand. The nucleic acid may be synthesized using oligonucleotide analogs or derivatives (e.g., inosine or phosphorothioate nucleotides). Such oligonucleotides can be used, for example, to prepare nucleic acids that have altered base-pairing abilities or increased resistance to nucleases.

An “isolated nucleic acid” is a DNA or RNA that is not immediately contiguous with both of the coding sequences with which it is immediately contiguous (one on the 5′ end and one on the 3′ end) in the naturally occurring genome of the organism from which it is derived. Thus, in one embodiment, an isolated nucleic acid includes some or all of the 5′ non-coding (e.g., promoter) sequences that are immediately contiguous to the coding sequence. The term therefore includes, for example, a recombinant DNA that is incorporated into a vector, into an autonomously replicating plasmid or virus, or into the genomic DNA of a prokaryote or eukaryote, or which exists as a separate molecule (e.g., a cDNA or a genomic DNA fragment produced by PCR or restriction endonuclease treatment), independent of other sequences. It also includes a recombinant DNA that is part of a hybrid gene encoding an additional polypeptide sequence.

The term “isolated” can refer to a nucleic acid or polypeptide that is substantially free of cellular material, viral material, or culture medium (when produced by recombinant DNA techniques), or chemical precursors or other chemicals (when chemically synthesized). Moreover, an “isolated nucleic acid fragment” is a nucleic acid fragment that is not naturally occurring as a fragment and would not be found in the natural state.

The term “oligonucleotide” refers to a nucleic acid sequence of at least about six nucleotides to about 100 nucleotides, for example, about 15 to 30 nucleotides, or about 20 to 25 nucleotides, which can be used, for example, as a primer in a PCR amplification or as a probe in a hybridization assay or in a microarray. Oligonucleotides may be natural or synthetic, e.g., DNA, RNA, modified backbones, etc.

The phrase “functionally active derivative” shall be given its ordinary meaning and shall include naturally-occurring or synthetic fragments, variants, and analogs that exhibit at least one function that is substantially similar to that of the compound from which it is derived. Functionally active derivatives may be, but need not be, structurally or chemically similar to the compound from which they are derived.

The term “stringent” as used here refers to hybridization conditions that are commonly understood in the art to define the commodities of the hybridization procedure. Stringency conditions can be low, high or medium, as those terms are commonly know in the art and well recognized by one of ordinary skill. High stringency hybridization conditions that will permit homologous nucleotide sequences to hybridize to a nucleotide sequence as given herein are well known in the art. As one example, hybridization of such sequences to the nucleic acid molecules disclosed herein can be carried out in 25% formamide, 5×SSC, 5×Denhardt's solution and 5% dextran sulfate at 42° C., with wash conditions of 25% formamide, 5×SSC and 0.1% SDS at 42° C., to allow hybridization of sequences of about 60% homology. Another example includes hybridization conditions of 6×SSC, 0.1% SDS at about 45° C., followed by wash conditions of 0.2×SSC, 0.1% SDS at 50-65° C. Another example of stringent conditions is represented by a wash stringency of 0.3 M NaCl, 0.03M sodium citrate, 0.1% SDS at 60-70° C. using a standard hybridization assay (see SAMBROOK et al., EDS., MOLECULAR CLONING: A LABORATORY MANUAL 2d ed. (Cold Spring Harbor, NY 1989, the entire contents of which are incorporated by reference herein). In various embodiments, stringent conditions can include, for example, highly stringent (i.e., high stringency) conditions (e.g., hybridization to filter-bound DNA in 0.5 M NaHPO₄, 7% sodium dodecyl sulfate (SDS), 1 mM EDTA at 65° C., and washing in 0.1×SSC/0.1% SDS at 68° C.), and/or moderately stringent (i.e., medium stringency) conditions (e.g., washing in 0.2×SSC/0.1% SDS at 42° C.).

Where a particular nucleotide sequence is said to have a specific percent identity to a reference nucleotide sequence, the percent identity is relative to the reference nucleotide sequence. For example, a nucleotide sequence that is 50%, 75%, 85%, 90%, 95% or 99% identical to a reference nucleotide sequence that is 100 bases long can have 50, 75, 85, 90, 95 or 99 bases that are completely identical to a 50, 75, 85, 90, 95 or 99 nucleotide sequence of the reference nucleotide sequence. The nucleotide sequence can also be a 100 base long nucleotide sequence that is 50%, 75%, 85%, 90%, 95% or 99% identical to the reference nucleotide sequence over its entire length. Of course, there are other nucleotide sequences that will also meet the same criteria.

A nucleic acid sequence that is “substantially identical” to a VKOR nucleotide sequence is at least 80%, 85% 90%, 95% or 99% identical to the nucleotide sequence of SEQ ID NO:8 or 9. For purposes of comparison of nucleic acids, the length of the reference nucleic acid sequence will generally be at least 40 nucleotides, e.g., at least 60 nucleotides or more nucleotides. Sequence identity can be measured using sequence analysis software (e.g., Sequence Analysis Software Package of the Genetics Computer Group, University of Wisconsin Biotechnology Center, 1710 University Avenue, Madison, Wis. 53705).

As is known in the art, a number of different programs can be used to identify whether a nucleic acid or amino acid has sequence identity or similarity to a known sequence. Sequence identity or similarity may be determined using standard techniques known in the art, including, but not limited to, the local sequence identity algorithm of Smith & Waterman, Adv. Appl. Math. 2, 482 (1981), by the sequence identity alignment algorithm of Needleman & Wunsch, J. Mol. Biol. 48,443 (1970), by the search for similarity method of Pearson & Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Drive, Madison, WI), the Best Fit sequence program described by Devereux et al., Nucl. Acid Res. 12, 387-395 (1984), preferably using the default settings, or by inspection.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35, 351-360 (1987); the method is similar to that described by Higgins & Sharp, CAB/OS 5:151-153 (1989).

Another example of a useful algorithm is the BLAST algorithm, described in Altschul et al., J. Mol. Biol. 215, 403-410, (1990) and Karlin et al., Proc. Natl. Acad. Sci. USA 90, 5873-5787 (1993). A particularly useful BLAST program is the WU-BLAST-2 program that was obtained from Altschul et al., Methods in Enzymology, 266, 460-480 (1996). WU-BLAST-2 uses several search parameters, which are preferably set to the default values. The parameters are dynamic values and are established by the program itself depending upon the composition of the particular sequence and composition of the particular database against which the sequence of interest is being searched; however, the values may be adjusted to increase sensitivity. An additional useful algorithm is gapped BLAST as reported by Altschul et al. Nucleic Acids Res. 25, 3389-3402.

The CLUSTAL program can also be used to determine sequence similarity. This algorithm is described by Higgins et al. (1988) Gene 73:237; Higgins et al. (1989) CABIOS 5:151-153; Corpet et al. (1988) Nucleic Acids Res. 16: 10881-90; Huang et al. (1992) CABIOS 8: 155-65; and Pearson et al. (1994) Meth. Mol. Biol. 24: 307-331.

In addition, for sequences that contain either more or fewer nucleotides than the nucleic acids disclosed herein, it is understood that in one embodiment, the percentage of sequence identity will be determined based on the number of identical nucleotides in relation to the total number of nucleotide bases. Thus, for example, sequence identity of sequences shorter than a sequence specifically disclosed herein will be determined using the number of nucleotide bases in the shorter sequence, in one embodiment. In percent identity calculations, relative weight is not assigned to various manifestations of sequence variation, such as, insertions, deletions, substitutions, etc.

The VKOR polypeptides of the invention include, but are not limited to, recombinant polypeptides, synthetic peptides and natural polypeptides. The invention also encompasses nucleic acid sequences that encode forms of VKOR polypeptides in which naturally occurring amino acid sequences are altered or deleted. Preferred nucleic acids encode polypeptides that are soluble under normal physiological conditions. Also within the invention are nucleic acids encoding fusion proteins in which all or a portion of VKOR is fused to an unrelated polypeptide (e.g., a marker polypeptide or a fusion partner) to create a fusion protein. For example, the polypeptide can be fused to a hexa-histidine tag to facilitate purification of bacterially expressed polypeptides, or to a hemagglutinin tag to facilitate purification of polypeptides expressed in eukaryotic cells, or to an HPC4 tag to facilitate purification of polypeptides by affinity chromatography or immunoprecipitation. The invention also includes isolated polypeptides (and the nucleic acids that encode these polypeptides) that include a first portion and a second portion; the first portion includes, e.g., all or a portion of a VKOR polypeptide, and the second portion includes, e.g., a detectable marker.

The fusion partner can be, for example, a polypeptide that facilitates secretion, e.g., a secretory sequence. Such a fused polypeptide is typically referred to as a preprotein. The secretory sequence can be cleaved by the cell to form the mature protein. Also within the invention are nucleic acids that encode VKOR fused to a polypeptide sequence to produce an inactive preprotein. Preproteins can be converted into the active form of the protein by removal of the inactivating sequence.

The invention also includes nucleic acids that hybridize, e.g., under stringent hybridization conditions (as defined herein) to all or a portion of the nucleotide sequence of SEQ ID NOS: 1-6, 8 or 9 or their complements. In particular embodiments, the hybridizing portion of the hybridizing nucleic acid is typically at least 15 (e.g., 20, 30, or 50) nucleotides in length. The hybridizing portion of the hybridizing nucleic acid is at least 80%, e.g., at least 95%, at least 98% or 100%, identical to the sequence of a portion or all of a nucleic acid encoding a VKOR polypeptide. Hybridizing nucleic acids of the type described herein can be used, for example, as a cloning probe, a primer (e.g., a PCR primer), or a diagnostic probe. Also included within the invention are small inhibitory RNAs (siRNAs) and/or antisense RNAs that inhibit the function of VKOR, as determined, for example, in an activity assay, as described herein and as is known in the art.

In another embodiment, the invention features cells, e.g., transformed cells, which contain a nucleic acid of this invention. A “transformed cell” is a cell into which (or into an ancestor of which) has been introduced, by means of recombinant nucleic acid techniques, a nucleic acid encoding all or a part of a VKOR polypeptide, and/or an antisense nucleic acid or siRNA. Both prokaryotic and eukaryotic cells are included, e.g., bacteria, yeast, insect, mouse, rat, human, plant and the like.

The invention also features nucleic acid constructs (e.g., vectors and plasmids) that include a nucleic acid of the invention that is operably linked to a transcription and/or translation control elements to enable expression, e.g., expression vectors. By “operably linked” is meant that a selected nucleic acid, e.g., a DNA molecule encoding a VKOR polypeptide, is positioned adjacent to one or more regulatory elements, e.g., a promoter, which directs transcription and/or translation of the sequence such that the regulatory elements can control transcription and/or translation of the selected nucleic acid.

In other embodiments, the present invention further provides fragments or oligonucleotides of the nucleic acids of this invention, which can be used as primers or probes. Thus, in some embodiments, a fragment or oligonucleotide of this invention is a nucleotide sequence that is at least 10, 15, 20, 25, 30, 35, 40, 45, 50, 55, 60, 65, 70, 75, 80, 85, 90, 100, 125, 150, 175, 200, 250, 300, 350, 400, 450, 500, 550, 600, 650, 700, 750, 800, 850, 900, 1000, 1500, 2000, 2500 or 3000 contiguous nucleotides of the nucleotide sequence set forth in SEQ ID NO:8 or SEQ ID NO:9. Examples of oligonucleotides of this invention are provided in the Sequence Listing included herewith. Such fragments or oligonucleotides can be detectably labeled or modified, for example, to include and/or incorporate a restriction enzyme cleavage site when employed as a primer in an amplification (e.g., PCR) assay.

Several embodiments of the invention comprise purified or isolated VKOR polypeptides, such as, for example, a polypeptide comprising, consisting essentially of and/or consisting of the amino acid sequence of SEQ ID NO:10 or a biologically active fragment or peptide thereof. Such fragments or peptides are typically at least about ten amino acids of the amino acid sequence of SEQ ID NO:10 (e.g., 15, 20, 25, 30, 35, 40, 45, 50, 55, 60, 65, 75, 85, 95, 100, 125, or 150 amino acids of the amino acid sequence of SEQ ID NO:10) and can be peptides or fragment of contiguous amino acids of the amino acid sequence of the VKOR protein (e.g., as set forth in SEQ ID NO:10). The biological activity of a fragment or peptide of this invention can be determined according to the methods provided herein and as are known in the art for identifying VKOR activity. The fragments and peptides of the VKOR protein of this invention can also be active as antigens for the production of antibodies. The identification of epitopes on a fragment or peptide of this invention is carried out by well known protocols and would be within the ordinary skill of one in the art.

As used herein, both “protein” and “polypeptide” mean any chain of amino acids, regardless of length or post-translational modification (e.g., glycosylation, phosphorylation or N-myristylation). Thus, the term “VKOR polypeptide” includes full-length, naturally occurring VKOR proteins, respectively, as well as recombinantly or synthetically produced polypeptides that correspond to a full-length, naturally occurring VKOR protein, or to a portion of a naturally occurring or synthetic VKOR polypeptide.

A “purified” or “isolated” compound or polypeptide is a composition that is at least 60% by weight the compound of interest, e.g., a VKOR polypeptide or antibody that is separated or substantially free from at least some of the other components of the naturally occurring organism or virus, for example, the cell or viral structural components or other polypeptides or nucleic acids commonly found associated with the polypeptide. As used herein, the “isolated” polypeptide is at least about 25%, 50%, 60%, 70%, 75%, 80%, 85%, 90%, 95%, 97%, 98%, 99% or more pure (w/w). Preferably the preparation is at least 75% (e.g., at least 90% or 99%) by weight the compound of interest. Purity can be measured by any appropriate standard method, e.g., column chromatography, polyacrylamide gel electrophoresis, or HPLC analysis.

Preferred VKOR polypeptides include a sequence substantially identical to all or a portion of a naturally occurring VKOR polypeptide. Polypeptides “substantially identical” to the VKOR polypeptide sequences described herein have an amino acid sequence that is at least 80% or 85% (e.g., 90%, 95% or 99%) identical to the amino acid sequence of the VKOR polypeptides of SEQ ID NO: 10. For purposes of comparison, the length of the reference VKOR polypeptide sequence will generally be at least 16 amino acids, e.g., at least 20, 25, 30, 35, 40, 45, 50, 75, or 100 amino acids.

In the case of polypeptide sequences that are less than 100% identical to a reference sequence, the non-identical positions are preferably, but not necessarily, conservative substitutions for the reference sequence. Conservative substitutions typically include, but are not limited to, substitutions within the following groups: glycine and alanine; valine, isoleucine, and leucine; aspartic acid and glutamic acid; asparagine and glutamine; serine and threonine; lysine and arginine; and phenylalanine and tyrosine.

Where a particular polypeptide is said to have a specific percent identity to a reference polypeptide of a defined length, the percent identity is relative to the reference polypeptide. Thus, for example, a polypeptide that is 50%, 75%, 85%, 90%, 95% or 99% identical to a reference polypeptide that is 100 amino acids long can be a 50, 75, 85, 90, 95 or 99 amino acid polypeptide that is completely identical to a 50, 75, 85, 90, 95 or 99 amino acid long portion of the reference polypeptide. It can also be a 100 amino acid long polypeptide that is 50%, 75%, 85%, 90%, 95% or 99% identical to the reference polypeptide over its entire length. Of course, other polypeptides also will meet the same criteria.

In one embodiment, the invention also comprises purified or isolated antibodies that specifically bind to a VKOR polypeptide of this invention or to a fragment thereof. By “specifically binds” is meant that an antibody recognizes and binds a particular antigen, e.g., a VKOR polypeptide, or an epitope on a fragment or peptide of a VKOR polypeptide, but does not substantially recognize and bind other molecules in a sample. In one embodiment the antibody is a monoclonal antibody and in other embodiments, the antibody is a polyclonal antibody. The production of both monoclonal and polyclonal antibodies, including chimeric antibodies, humanized antibodies, single chain antibodies, bi-specific antibodies, antibody fragments, etc., is well known in the art.

In another aspect, the invention comprises a method for detecting a VKOR polypeptide in a sample. This method comprises contacting the sample with an antibody that specifically binds a VKOR polypeptide or a fragment thereof under conditions that allow the formation of a complex between an antibody and VKOR; and detecting the formation of a complex, if any, as detection of a VKOR polypeptide or fragment thereof in the sample. Such immunoassays are well known in the art and include immunoprecipitation assays, immunoblotting assays, immunolabeling assays, ELISA, etc.

In another embodiment, the present invention further provides a method of detecting a nucleic acid encoding a VKOR polypeptide in a sample, comprising contacting the sample with a nucleic acid of this invention that encodes VKOR or a fragment thereof, or a complement of a nucleic acid that encodes VKOR or a fragment thereof, under conditions whereby a hybridization complex can form, and detecting formation of a hybridization complex, thereby detecting a nucleic acid encoding a VKOR polypeptide in a sample. Such hybridization assays are well known in the art and include probe detection assays and nucleic acid amplification assays.

Also encompassed by one embodiment of the invention is a method of obtaining a gene related to (i.e., a functional homologue of) the VKOR gene. Such a method entails obtaining or producing a detectably-labeled probe comprising an isolated nucleic acid which encodes all or a portion of VKOR, or a homolog thereof; screening a nucleic acid fragment library with the labeled probe under conditions that allow hybridization of the probe to nucleic acid fragments in the library, thereby forming nucleic acid duplexes; isolating labeled duplexes, if any; and preparing a full-length gene sequence from the nucleic acid fragments in any labeled duplex to obtain a gene related to the VKOR gene.

A further aspect of the present invention is a method of making a vitamin K dependent protein, comprising culturing a cell that expresses a nucleic acid encoding a vitamin K dependent protein that, in the presence of vitamin K, produces a vitamin K dependent protein; and then harvesting the vitamin K dependent protein from the culture medium, wherein the cell comprises and expresses an exogenous nucleic acid encoding vitamin K epoxide reductase (VKOR), thereby producing VKOR and in some embodiments the cell further comprises and expresses an exogenous nucleic acid encoding vitamin K dependent carboxylase, thereby producing vitamin K dependent carboxylase as described herein. In some embodiments, the expression of the VKOR-encoding nucleic acid and the production of the VKOR causes the cell to produce greater levels of the vitamin K dependent protein and/or greater levels of active (e.g., fully carboxylated) vitamin K dependent protein than would be produced in the absence of the VKOR or in the absence of the VKOR and carboxylase.

Thus, in some embodiments, the present invention also provides a method of producing a vitamin K dependent protein, comprising:
a) introducing into a cell a nucleic acid that encodes a vitamin K dependent protein under conditions whereby the nucleic acid is expressed and the vitamin K dependent protein is produced in the presence of vitamin K, wherein the cell comprises a heterologous nucleic acid encoding vitamin K dependent carboxylase and further comprises a heterologous nucleic acid encoding vitamin K epoxide reductase; and
b) optionally collecting the vitamin K dependent protein from the cell.

In one embodiment, the present invention also provides a method of increasing the amount of carboxylated vitamin K dependent protein in a cell, comprising introducing into a cell that expresses a first nucleic acid encoding a vitamin K dependent protein a second heterologous nucleic acid encoding vitamin K epoxide reductase (VKOR) under conditions whereby said first and second nucleic acids are expressed to produce a vitamin K dependent protein and VKOR, respectively.

Further provided herein is a method of increasing the carboxylation of a vitamin K dependent protein, comprising introducing into a cell that expresses a first nucleic acid encoding a vitamin K dependent protein a second heterologous nucleic acid encoding vitamin K epoxide reductase (VKOR) under conditions whereby said first and second nucleic acids are expressed to produce a vitamin K dependent protein and VKOR, respectively.

In addition, in another embodiment, the present invention provides a method of producing a carboxylated (e.g., fully carboxylated) vitamin K dependent protein in a cell, comprising introducing into a cell that expresses a first nucleic acid encoding a vitamin K dependent protein a second heterologous nucleic acid encoding vitamin K epoxide reductase (VKOR) under conditions whereby said first and second nucleic acids are expressed to produce a vitamin K dependent protein and VKOR, respectively, wherein the amount of carboxylated vitamin K dependent protein produced in the cell in the presence of VKOR is increased as compared to the amount of carboxylated vitamin K dependent protein produced in the cell in the absence of VKOR.

Furthermore, in another embodiment, the present invention provides a method of producing a vitamin K dependent protein in a cell, comprising introducing into a cell that expresses a first nucleic acid encoding a vitamin K dependent protein a second exogenous nucleic acid encoding vitamin K epoxide reductase (VKOR) under conditions whereby said first and second nucleic acids are expressed to produce a vitamin K dependent protein and VKOR, respectively, wherein 100%, 90%, 80%, 70% or 60% of the vitamin K dependent protein produced in the cell in the presence of VKOR is carboxylated (e.g., fully carboxylated).

Also included herein is a method of producing a vitamin K dependent protein in a cell, comprising introducing into a cell that expresses a first nucleic acid encoding a vitamin K dependent protein a second heterologous nucleic acid encoding vitamin K epoxide reductase (VKOR) under conditions whereby said first and second nucleic acids are expressed to produce a vitamin K dependent protein and VKOR, respectively.

The present invention further comprises embodiments wherein nucleic acid encoding vitamin K epoxide reductase can be introduced into a cell to improve the growth characteristics (e.g., enhance growth rate; increase survival time, etc.) of the cell.

In some embodiments of this invention, the nucleic acid encoding vitamin K epoxide reductase can be a nucleic acid that is naturally present in the cell (i.e., endogenous to the cell). In other embodiments, the nucleic acid encoding vitamin K epoxide reductase can be an exogenous nucleic acid that is introduced into the cell. In further embodiments, the cell of this invention can comprise an endogenous nucleic acid encoding vitamin K epoxide reductase and an exogenous nucleic acid encoding vitamin K epoxide reductase.

In some embodiments of the methods described above, the cell can further comprise a third nucleic acid encoding a vitamin K dependent carboxylase, which can be, but is not limited to, a bovine vitamin K dependent carboxylase. In particular embodiments, the vitamin K-dependent carboxylase is vitamin K gamma glutamyl carboxylase (VKGC). The VKGC used in the methods of this invention can be VKGC from any vertebrate or invertebrate species that produces VKGC, as are known in the art.

According to several embodiments, in methods of this invention where the amount of carboxylated vitamin K-dependent protein is increased in a cell in the presence of VKOR and/or VKGC, the amount of carboxylated or fully carboxylated vitamin K dependent protein produced in the cell in the presence of VKOR and/or VKGC can be increased 10%, 20%, 30%, 40%, 50%, 60%, 70%, 80%, 90% 100% 125% 150%, 200% or 300%, as compared to the amount of carboxylated or fully carboxylated vitamin K dependent protein produced in the cell in the absence of VKOR and/or VKGC.

By “fully carboxylated” in some embodiments is meant that all sites (or in some embodiments, the majority of sites) on a vitamin K dependent protein that can undergo carboxylation are carboxylated. In some embodiments, fully carboxylated can mean that all vitamin K dependent proteins are carboxylated to some extent and/or that all vitamin K dependent proteins are carboxylated at all or at the majority of carboxylation sites. A carboxylated vitamin K dependent protein or fully carboxylated vitamin K dependent protein is an active protein. By “active protein” is meant that the vitamin K dependent protein has or is capable of activity in carrying out its biological function (e.g., an enzymatic activity for factor IX or factor X).

The vitamin K dependent protein that can be produced according to the methods of this invention can be any vitamin K dependent protein now known or later identified as such, including but not limited to, Factor VII, activated Factor VII (Factor VIIA), Factor IX, Factor X, Protein C, activated Protein C, Protein S, bone Gla protein (osteocalcin), matrix Gla protein and prothrombin, including modified versions of such proteins as described herein, in any combination. The nucleotide sequences and amino acid sequences of the vitamin K dependent proteins of this invention are known in the art. Nonlimiting examples of sequences of some of the vitamin K dependent proteins of this invention include human factor VII, which has GenBank Accession No. BC130468, human factor VIIa, which has SwissProt Accession No. P08709 and Protein C, which has GenBank Accession No. NM_000312. These examples demonstrate the availability of these sequences in the art and are not intended to be limiting in any way, as the present invention includes any vitamin K dependent protein, in any combination.

Any cell that can be transformed with the nucleic acids described herein can be used as described herein, although in some embodiments non-human or even non-mammalian cells can be used. Thus, a cell or cell line of this invention can be, for example, a human cell, an animal cell, a plant cell and/or an insect cell. Nucleic acids encoding vitamin K dependent carboxylase and nucleic acids encoding vitamin K dependent proteins as described herein are well known in the art and their introduction into cells for expression would be carried out according to routine protocols. Thus, in some embodiments, the present invention provides a cell that comprises a nucleic acid (either endogenous or exogenous to the cell) that encodes a vitamin K dependent protein. The vitamin K dependent protein is produced in the cell in the presence of vitamin K. The cell further comprises a heterologous (i.e., exogenous) nucleic acid encoding vitamin K epoxide reductase (VKOR) and/or a vitamin K dependent carboxylase. The cell can be maintained under conditions known in the art whereby the nucleic acid encoding VKOR and/or the vitamin K dependent carboxylase are expressed and VKOR and/or the carboxylase are produced in the cell.

Certain embodiments of this invention are based on the inventors' discovery that a subject's therapeutic dose of warfarin for anticoagulation therapy can be correlated with the presence of one or more single nucleotide polymorphisms in the VKOR gene of the subject. Thus, the present invention also provides a method of identifying a human subject having increased or decreased sensitivity to warfarin, comprising detecting in the subject the presence of a single nucleotide polymorphism (SNP) in the VKOR gene, wherein the single nucleotide polymorphism is correlated with increased or decreased sensitivity to warfarin, thereby identifying the subject as having increased or decreased sensitivity to warfarin.

An example of a SNP correlated with an increased sensitivity to warfarin is a G→C alteration at nucleotide 2581 (SEQ ID NO:12) (in intron 2 of the VKOR gene; GenBank accession no. refSNP ID: rs8050894, incorporated by reference herein) of the nucleotide sequence of SEQ ID NO:11, which is a reference sequence encompassing the genomic sequence of SEQ ID NO:8 and approximately 1000 nucleotides preceding and following this sequence. This sequence can be located as having the genome position “human chromosome 16p11.2” or in the physical map in the NCBI database as human chromosome 16: 31009700-31013800.

Examples of SNPs correlated with a decreased sensitivity to warfarin are a T→C alteration at nucleotide 3294 (SEQ ID NO:13) (in intron 2 of the VKOR gene; GenBank accession no. refSNP ID: rs2359612, incorporated by reference herein) of the nucleotide sequence of SEQ ID NO:11 and a G→A alteration at nucleotide 4769 (SEQ ID NO:14) (in the 3′ UTR of the VKOR gene; GenBank accession no. refSNP ID: rs7294, incorporated by reference herein) of the nucleotide sequence of SEQ ID NO:11.

As used herein, a subject having an “increased sensitivity to warfarin” is a subject for whom a suitable therapeutic or maintenance dose of warfarin is lower than the therapeutic or maintenance dose of warfarin that would suitable for a normal subject, i.e., a subject who did not carry a SNP in the VKOR gene that imparts a phenotype of increased sensitivity to warfarin. Conversely, as used herein, a subject having a “decreased sensitivity to warfarin” is a subject for whom a suitable therapeutic or maintenance dose of warfarin is higher than the therapeutic or maintenance dose of warfarin that would suitable for a normal subject, i.e., a subject who did not carry a SNP in the VKOR gene that imparts a phenotype of decreased sensitivity to warfarin. An example of a typical therapeutic dose of warfarin for a normal subject is 35 mg per week, although this amount can vary (e.g., a dose range of 3.5 to 420 mg per week is described in Aithal et al. (1999) Lancet 353:717-719). A typical therapeutic dose of warfarin can be determined for a given study group according to the methods described herein, which can be used to identify subjects with therapeutic warfarin doses above or below this dose, thereby identifying subjects having decreased or increased sensitivity to warfarin.

Further provided herein is a method of identifying a human subject having increased or decreased sensitivity to warfarin, comprising: a) correlating the presence of a single nucleotide polymorphism in the VKOR gene with increased or decreased sensitivity to warfarin; and b) detecting the single nucleotide polymorphism of step (a) in the subject, thereby identifying a subject having increased or decreased sensitivity to warfarin.

In additional embodiments, the present invention provides a method of identifying a single nucleotide polymorphism in the VKOR gene correlated with increased or decreased sensitivity to warfarin, comprising: a) identifying a subject having increased or decreased sensitivity to warfarin; b) detecting in the subject the presence of a single nucleotide polymorphism in the VKOR gene; and c) correlating the presence of the single nucleotide polymorphism of step (b) with the increased or decreased sensitivity to warfarin in the subject, thereby identifying a single nucleotide polymorphism in the VKOR gene correlated with increased or decreased sensitivity to warfarin.

Also provided herein as another embodiment is a method of correlating a single nucleotide polymorphism in the VKOR gene of a subject with increased or decreased sensitivity to warfarin, comprising: a) identifying a subject having increased or decreased sensitivity to warfarin; b) determining the nucleotide sequence of the VKOR gene of the subject of (a); c) comparing the nucleotide sequence of step (b) with the wild type nucleotide sequence of the VKOR gene; d) detecting a single nucleotide polymorphism in the nucleotide sequence of (b); and e) correlating the single nucleotide polymorphism of (d) with increased or decreased sensitivity to warfarin in the subject of (a).

A subject is identified as having an increased or decreased sensitivity to warfarin by establishing a therapeutic or maintenance dose of warfarin for anticoagulation therapy according to well known protocols and comparing the therapeutic or maintenance dose for that subject with the therapeutic or maintenance dose of warfarin for anticoagulation therapy of a population of normal subjects (e.g., subjects lacking any SNPs in the VKOR gene correlated with increased or decreased sensitivity to warfarin) from which an average or mean therapeutic or maintenance dose of warfarin is calculated. A subject having a therapeutic or maintenance dose of warfarin that is below the average therapeutic or maintenance dose of warfarin (e.g., the dose of warfarin that is therapeutic or provides a maintenance level for a subject that has a wild type VKOR gene, i.e., lacking any single nucleotide polymorphisms associated with warfarin sensitivity) is a subject identified as having an increased sensitivity to warfarin. A subject having a therapeutic or maintenance dose of warfarin that is above the average therapeutic or maintenance of warfarin is a subject identified as having a decreased sensitivity to warfarin. An average therapeutic or maintenance dose of warfarin for a subject with a wild type VKOR gene would be readily determined by one skilled in the art.

The nucleotide sequence of the VKOR gene of a subject is determined according to methods standard in the art, and as described in the Examples provided herein. For example, genomic DNA is extracted from cells of a subject and the VKOR gene is located and sequenced according to known protocols. Single nucleotide polymorphisms in the VKOR gene are identified by a comparison of a subject's sequence with the wild type sequence as known in the art (e.g., the reference sequence as shown herein as SEQ ID NO:11).

A SNP in the VKOR gene is correlated with an increased or decreased sensitivity to warfarin by identifying the presence of a SNP or multiple SNPs in the VKOR gene of a subject also identified as having increased or decreased sensitivity to warfarin, i.e., having a maintenance or therapeutic dose of warfarin that is above or below the average dose and performing a statistical analysis of the association of the SNP or SNPs with the increased or decreased sensitivity to warfarin, according to well known methods of statistical analysis. An analysis that identifies a statistical association (e.g., a significant association) between the SNP(s) (genotype) and increased or decreased warfarin sensitivity (phenotype) establishes a correlation between the presence of the SNP(s) in a subject and an increased or decreased sensitivity to warfarin in that subject.

It is contemplated that a combination of factors, including the presence of one or more SNPs in the VKOR gene of a subject, can be correlated with an increased or decreased sensitivity to warfarin in that subject. Such factors can include, but are not limited to cytochrome p450 2C9 polymorphisms, race, age, gender, smoking history and hepatic disease.

Thus, in a further embodiment, the present invention provides a method of identifying a human subject having increased or decreased sensitivity to warfarin, comprising identifying in the subject the presence of a combination of factors correlated with an increased or decreased sensitivity to warfarin selected from the group consisting of one or more single nucleotide polymorphisms of the VKOR gene, one or more cytochrome p450 2C9 polymorphisms, race, age, gender, smoking history, hepatic disease and any combination of two or more of these factors, wherein the combination of factors is correlated with increased or decreased sensitivity to warfarin, thereby identifying the subject having increased or decreased sensitivity to warfarin.

Further provided herein is a method of identifying a human subject having increased or decreased sensitivity to warfarin, comprising: a) correlating the presence of a combination of factors with an increased or decreased sensitivity to warfarin, wherein the factors are selected from the group consisting of one or more single nucleotide polymorphisms of the VKOR gene, one or more cytochrome p450 2C9 polymorphisms, race, age, gender, smoking history, hepatic disease and any combination of two or more of these factors; and b) detecting the combination of factors of step (a) in the subject, thereby identifying a subject having increased or decreased sensitivity to warfarin.

In additional embodiments, the present invention provides a method of identifying a combination of factors correlated with an increased or decreased sensitivity to warfarin, wherein the factors are selected from the group consisting of one or more single nucleotide polymorphisms of the VKOR gene, one or more cytochrome p450 2C9 polymorphisms, race, age, gender, smoking history, hepatic disease and any combination of two or more of these factors, comprising: a) identifying a subject having increased or decreased sensitivity to warfarin; b) detecting in the subject the presence of a combination of the factors; and c) correlating the presence of the combination of factors of step (b) with the increased or decreased sensitivity to warfarin in the subject, thereby identifying a combination of factors correlated with increased or decreased sensitivity to warfarin.

Also provided herein is a method of correlating a combination of factors, wherein the factors are selected from the group consisting of one or more single nucleotide polymorphisms of the VKOR gene, one or more cytochrome p450 2C9 polymorphisms, race, age, gender, smoking history, hepatic disease and any combination of two or more of these factors, with increased or decreased sensitivity to warfarin, comprising: a) identifying a subject having increased or decreased sensitivity to warfarin; b) identifying the presence of a combination of the factors in the subject; and c) correlating the combination of the factors of (b) with increased or decreased sensitivity to warfarin in the subject of (a).

A combination of factors as described herein is correlated with an increased or decreased sensitivity to warfarin by identifying the presence of the combination of factors in a subject also identified as having increased or decreased sensitivity to warfarin and performing a statistical analysis of the association of the combination of factors with the increased or decreased sensitivity to warfarin, according to well known methods of statistical analysis. An analysis that identifies a statistical association (e.g., a significant association) between the combination of factors and the warfarin sensitivity phenotype (increased or decreased) establishes a correlation between the presence of the combination of factors in a subject and an increased or decreased sensitivity to warfarin in that subject.

Further provided herein are nucleic acids encoding VKOR and comprising one or more SNPs as described herein. Thus, the present invention further provides nucleic acids comprising, consisting essentially of and/or consisting of the nucleotide sequence as set forth in SEQ ID NOs:12, 13, 14, 15 and 16. The nucleic acids can be present in a vector and the vector can be present in a cell. Further included are proteins encoded by a nucleic acid comprising a nucleotide sequence as set forth in SEQ ID NOs:12, 13, 14, 15 and 16, as well as antibodies that specifically bind a protein encoded by a nucleic acid comprising a nucleotide sequence as set forth in SEQ ID NOs:12, 13, 14, 15 and 16. The present invention is more particularly described in the following examples that are intended as illustrative only since numerous modifications and variations therein will be apparent to those skilled in the art.

### EXAMPLES

### Example I Correlation Between SNPS in VKOR Gene and Increased or Decreased Sensitivity to Warfarin

The most prevalent isoform of the VKOR gene is about 4 kb long, has three exons and encodes an enzyme of 163 amino acids with a mass of 18.4 kDa. In the present study, three mutations vk2581(G>C), vk3294(T>C) and vk4769(G>A), identified as SNPs (heterozygosity ratios of 46.9%, 46.8% and 46.3%, respectively) were examined for a correlation between their presence in a subject and the maintenance dose of warfarin required to achieve a therapeutically effective response.

### 1. Selection of Subjects

Subjects were obtained from the UNC Coagulation Clinic in the Ambulatory Care Center. Informed consent was obtained by a trained genetic counselor. Subjects not fluent in English were excluded because of the lack of translators and the requirement for consent. To qualify for the study, subjects had warfarin for at least six months, were older than 18 and were followed by the UNC Coagulation clinic at the Ambulatory Care Clinic.

2. Extraction of Genomic DNA from Whole Blood

Genomic DNAs were extracted from the whole blood of subjects using QIAamp DNA Blood Mini Kit (QIAGEN cat #51104). The DNA concentration was adjusted to 10 ng/μL.

### 3. Sequencing of the Genomic DNA Samples

Approximately 10 ng of DNA was used for polymerase chain reaction (PCR) assays. The primers used to amplify the VKOR gene were: Exon 1-5′ CCAATCGCCGAGTCAGAGG (SEQ ID NO:29) and Exon 1-3′ CCCAGTCCCCAGCACTGTCT (SEQ ID NO:30) for the 5′-UTR and Exon 1 region; Exon 2-5′ AGGGGAGGATAGGGTCAGTG (SEQ ID NO:31) and Exon 2-3′ CCTGTTAGTTACCTCCCCACA (SEQ ID NO:32) for the Exon 2 region; and Exon 3-5′ ATACGTGCGTAAGCCACCAC (SEQ ID NO:33) and Exon 3-3′ ACCCAGATATGCCCCCTTAG (SEQ ID NO:34) for the Exon3 and 3′-UTR region. Automated high throughput capillary electrophoresis DNA sequencing was used for detecting SNPs in the VKOR gene.

### 4. Detection of Known SNPs Using Real-Time PCR

The assay reagents for SNP genotyping were from the Assay-by-Design™ service (Applied Biosystems, cat #4332072). The primers and probes (FAM™ and VIC™ dye-labeled) were designed using Primer Express software and were synthesized in an Applied Biosystems synthesizer. The primer pairs for each SNP are located at the upstream/downstream position of the SNP site and can generate less than 100 bp length of a DNA fragment in the PCR reaction. The FAM™ and VIC™ dye-labeled probes were designed to cover the SNP sites with a length of 15-16 nt. The primer and probe sequences for each VKOR SNP are shown in Table 2.

The 2×TaqMan™ Universal PCR Master Mix, No AmpErase UNG (Applied Biosystems, cat #4324018) was used in the PCR reactions. Forty cycles of real-time PCR were performed in an Opticon II (MJ Research) machine. There was a 10 minute 95° C. preheat followed by 92° C. for 15 sec, 60° C. for 1 min. and then a plate reading. The results were read according to the signal value of FAM and VIC dye.

### 5. Statistical Analysis

The difference of average dose between different genotypes was compared by analysis of variance (ANOVA) using SAS version 8.0 (SAS, Inc., Cary, NC). A two-sided p value less than 0.05 was considered significant. Examination of the distribution and residuals for the average dose of treatment among the SNP groups indicated that a log transformation was necessary to satisfy the assumption of homogeneity of variance.

6. Correlation of SNPs with Warfarin Dosage

By direct genomic DNA sequencing and SNP real-time PCR detection, five SNPs were identified in the VKOR gene: one in the 5′-UTR, two in intron II, one in the coding region and one in the 3′-UTR (Table 1).

Among these SNPs, the vk563 and vk4501 SNPs allele were carried by only one of the 58 subjects of the study (a triple heterozygous, also carrying the 3′-UTR SNP allele), while the other SNPs were identified in 17-25 heterozygous patients.

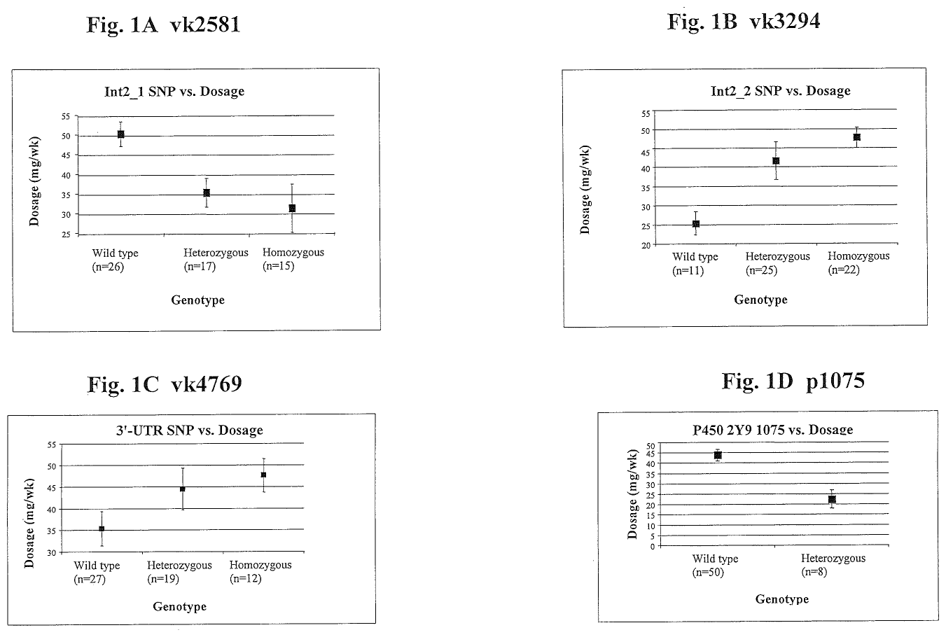

Each marker was first analyzed independently. FIG. 1A shows that the average warfarin dose for patients with the vk2581 wild type allele was 50.19±3.20 mg per week (n=26), while those heterozygous and homozygous for this polymorphism were 35.19±3.73 (n=17) and 31.14±6.2 mg per week (n=15), respectively. FIG. 1B shows that the average warfarin dose for patients with the wild-type vk3294 allele was 25.29±3.05 mg per week (n=11), while patients bearing the heterozygous and homozygous alleles were 41.68±4.92 (n=25) and 47.73±2.75 mg per week (n=22), respectively. FIG. 1C shows the average warfarin dose for patients with vk4769 SNP wild type was 35.35±4.01 mg per week (n=27), while patients with the heterozygous and homozygous alleles required 44.48±4.80 (n=19) and 47.56±3.86 mg per week (n=12), respectively. It was also observed that P450 2C9*3 has a significant effect on warfarin dose (FIG. 1D), as previously reported (Joffe et al. (2004) “Warfarin dosing and cytochrome P450 2C9 polymorphisms” Thromb Haemost 91:1123-1128). The average warfarin dose for patients with P450 2C9*1 (wild type) was 43.82±2.75 mg per week (n=50), while patients heterozygous for this allele required 22.4±4.34 mg per week (n=8).

### 7. Statistical Analysis

The association of the Logₑ (warfarin average dosage)(LnDose) with the SNPs in the VKOR gene was examined by analysis of variance (ANOVA). SAS was used first to do a repeated procedure in which a series of factors (race, gender, smoking history, hepatic diseases, SNPs at cytochrome P450 2Y9 gene, etc.) were examined to identify factors, excluding VKOR SNPs, which might affect dosage. P450 2C9*3 was significantly associated with the average dose of warfarin; thus, it was included as a covariant for further analysis. The analysis indicated that the three VKOR SNPs were still significantly associated with weekly warfarin dose (vk2581, P<0.0001; vk3294, P<0.0001; and vk4769, P=0.0044), when the covariance is included.

To specifically test if the three SNPs of VKOR were independently associated with warfarin dosage, the analysis was repeated in which two SNPs in the VKOR gene were included as covariates for the other SNP. The three VKOR SNPs are located within 2 kb distance of one another and are expected to be closely linked. It was clear from inspection that, at least for Caucasians, one haplotype (where A=vk2581 guanine and a=vk2581 cytosine; B=vk3294 thymine and b=vk3924 cytosine; C=vk4769 guanine and c=vk4769 adenine) was AAbbcc and another aaBBCC. The distribution of individual SNPs in patients was found to be significantly correlated with the others (R=0.63-0.87, p<0.001). Indeed, subjects with the haplotype AAbbcc (n=7) required a significantly higher dosage of warfarin (warfarin dosage=48.98±3.93) compared to those patients with haplotype aaBBCC (25.29±3.05; p<0.001).

### Example 2 siRNA Design and Synthesis

siRNAs were selected using an advanced version of a rational design algorithm (Reynolds et al. (2004) “Rational siRNA design for RNA interference” Nature Biotechnology 22:326-330). For each of the 13 genes, four siRNAs duplexes with the highest scores were selected and a BLAST search was conducted using the Human EST database. To minimize the potential for off-target silencing effects, only those sequence targets with more than three mismatches against un-related sequences were selected (Jackson et al. (2003) “Expression profiling reveals off-target gene regulation by RNAi” Nat Biotechnol 21:635-7). All duplexes were synthesized in Dharmacon (Lafayette, CO) as 21-mers with UU overhangs using a modified method of 2′-ACE chemistry (Scaringe (2000) “Advanced 5′-silyl-2′-orthoester approach to RNA oligonucleotide synthesis” Methods Enzymol 317:3-18) and the AS strand was chemically phosphorylated to ensure maximum activity (Martinez et al. (2002) “Single-stranded antisense siRNAs guide target RNA cleavage in RNAi” Cell 110:563-74).

### Example 3 siRNA Transfection

Transfection was essentially as previously described (Harborth et al. (2001) “Identification of essential genes in cultured mammalian cells using small interfering RNAs” J Cell Sci 114:4557-65) with minor modifications.

### Example 4 VKOR Activity Assay

siRNA transfected A549 cells were trypsinized and washed twice with cold PBS. 1.5×10⁷ cells were taken for each VKOR assay. 200 μL buffer D (250 mM Na₂HPO₄—NaH₂PO₄, 500 mM KCl, 20% glycerol and 0.75% CHAPS, pH 7.4) was added to the cell pellet, followed by sonication of the cell lysate. For assays of solubilized microsomes, microsomes were prepared from 2×10⁹ cells as described (Lin et al. (2002) “The putative vitamin K-dependent gamma-glutamyl carboxylase internal propeptide appears to be the propeptide binding site” J Biol Chem 277:28584-91); 10 to 50 μL of solubilized microsomes were used for each assay. Vitamin K epoxide was added to the concentration indicated in the figure legends and DTT was added to 4 mM to initiate the reaction. The reaction mixture was incubated in yellow light at 30° C. for 30 minutes and stopped by adding 500 μL 0.05 M AgNO₃: isopropanol (5:9). 500 μL hexane was added and the mixture was vortexed vigorously for 1 minute to extract the vitamin K and KO. After 5 minutes centrifugation, the upper organic layer was transferred to a 5-mL brown vial and dried with N₂. 150 μL HPLC buffer, acetonitrile:isopropanol:water (100:7:2), was added to dissolve the vitamin K and KO and the sample was analyzed by HPLC on an A C-18 column (Vydac, cat #218TP54).

### Example 5 RT-qPCR (Reverse Transcriptase Quantitative PCR)

1×10⁶ cells were washed with PBS twice and total RNA was isolated with Trizol reagent according to the manufacturer's protocol (Invitrogen). 1 μg of RNA was digested by RQ1 DNasel (Promega) and heat-inactivated. First strand cDNA was made with M-MLV reverse transcriptase (Invitrogen). cDNAs were mixed with DyNAmo SYBR Green qPCR pre-mix (Finnzymes) and real-time PCR was performed with an Opticon II PCR thermal cycler (MJ Research). The following primers were used:

13124769-5′ (F):
(TCCAACAGCATATTCGGTTGC, SEQ ID NO: 1);
13124769-3 (R)′:
(TTCTTGGACCTTCCGGAAACT, SEQ ID NO: 2);
GAPDH-F:
(GAAGGTGAAGGTCGGAGTC, SEQ ID NO: 3);
GAPDH-R:
(GAAGATGGTGATGGGATTTC, SEQ ID NO: 4);
Lamin-RT-F:
(CTAGGTGAGGCCAAGAAGCAA, SEQ ID NO: 5)
and
Lamin-RT-R:
(CTGTTCCTCTCAGCAGACTGC, SEQ ID NO: 6).

### Example 6 Over-Expression of VKOR in Sf9 Insect Cell Line

The cDNA for the mGC11276 coding region was cloned into pVL1392 (Pharmingen), with the HPC4 tag (EDQVDPRLIDGK, SEQ ID NO: 7) at its amino terminus and expressed in Sf9 cells as described (Li et al. (2000) “Identification of a Drosophila vitamin K-dependent gamma-glutamyl carboxylase” J Biol Chem 275:18291-6).

### Example 7 Gene Selection

The search for the VKOR gene was focused on human chromosome sixteen between markers D16S3131 and D16S419. This region corresponds to chromosome 16 at 50 cM-65 cM on the genetic map and 26-46.3 Mb on the physical map. 190 predicted coding sequences in this region were analyzed by a BLASTX search of the NCBI non-redundant protein database. Those human genes and orthologs from related species with known function were eliminated. Because VKOR appears to be a transmembrane protein (Carlisle & Suttie (1980) “Vitamin K dependent carboxylase: subcellular location of the carboxylase and enzymes involved in vitamin K metabolism in rat liver” Biochemistry 19:1161-7), the remaining genes were translated according to the cDNA sequences in the NCBI database and analyzed with the programs TMHMM and TMAP (Biology WorkBench, San Diego Supercomputer System) to predict those with transmembrane domains. Thirteen genes predicted to code for integral membrane proteins were chosen for further analysis.

### Example 8 Cell Line Screening for VKOR Activity

The strategy was to identify a cell line expressing relatively high amounts of VKOR activity and use siRNA to systematically knock down all thirteen candidate genes. siRNA, double stranded RNA of 21-23 nucleotides, has been shown to cause specific RNA degradation in cell culture (Hara et al. (2002) “Raptor, a binding partner of target of rapamycin (TOR), mediates TOR action” Cell 110:177-89; Krichevsky & Kosik (2002) “RNAi functions in cultured mammalian neurons” Proc Natl Acad Sci USA 99:11926-9; Burns et al. (2003) “Silencing of the Novel p53 Target Gene Snk/Plk2 Leads to Mitotic Catastrophe in Paclitaxel (Taxol)-Exposed Cells” Mol Cell Biol 23:5556-71). However, application of siRNA for large scale screening in mammalian cells has not previously been reported because of the difficulty in identifying a functional target for a specific mammalian cell mRNA (Holen et al. (2003) “Similar behaviour of single-strand and double-strand siRNAs suggests they act through a common RNAi pathway” Nucleic Acids Res 31:2401-7). The development of a rational selection algorithm (Reynolds et al.) for siRNA design increases the probability that a specific siRNA can be developed; furthermore, the probability of success can be increased by pooling four rationally selected siRNAs. Using siRNA to search for previously unidentified genes has the advantage that, even if VKOR activity requires the product of more than one gene for activity, the screen should still be effective because the assay determines the loss of enzymatic activity.

Fifteen cell lines were screened and a human lung carcinoma line, A549, was identified to exhibit sufficient warfarin-sensitive VKOR activity for facile measurement. A second human colorectal adenocarcinoma cell line, HT29, which expressed very little VKOR activity, was used as a reference.

### Example 9 siRNA Inhibition of VKOR Activity in A549 Cells

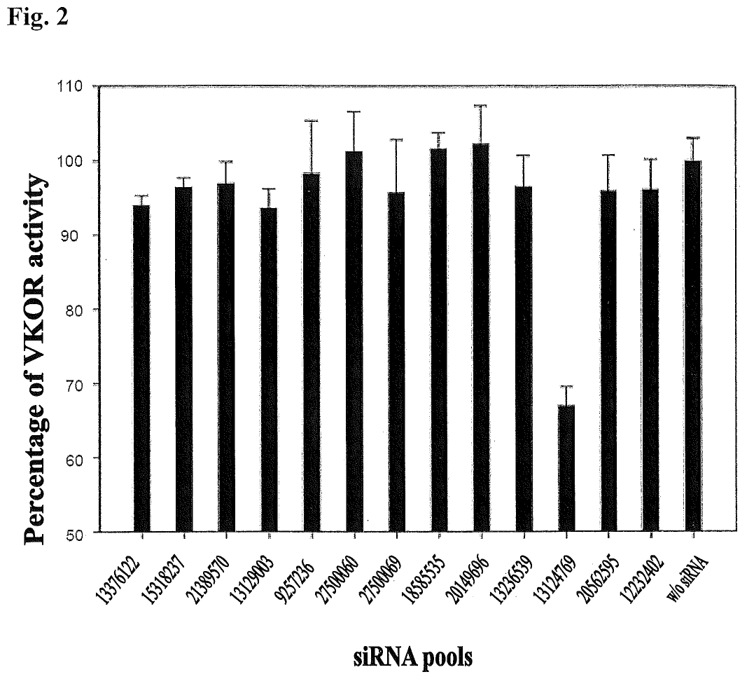

Each of the thirteen pools of siRNA were transfected in triplicate into A549 cells and assayed for VKOR activity after 72 hours. One siRNA pool specific for gene gi:13124769 reduced VKOR activity by 64%-70% in eight separate assays (FIG. 2).

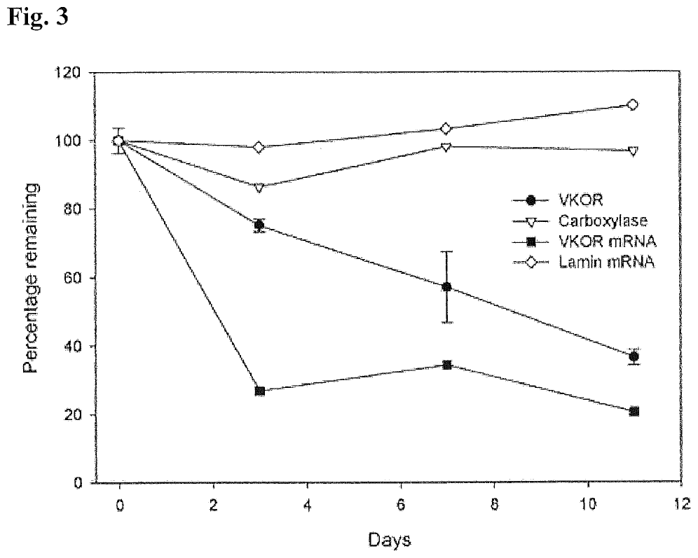

One possible reason that VKOR activity was inhibited to only ˜35% of its initial activity after 72 hours is that the half-life of mammalian proteins varies greatly (from minutes to days) (Zhang et al. (1996) “The major calpain isozymes are long-lived proteins. Design of an antisense strategy for calpain depletion in cultured cells” J Biol Chem 271:18825-30; Bohley (1996) “Surface hydrophobicity and intracellular degradation of proteins” Biol Chem 377:425-35; Dice & Goldberg (1975) “Relationship between in vivo degradative rates and isoelectric points of proteins” Proc Natl Acad Sci USA 72:3893-7), and mRNA translation is being inhibited, not enzyme activity. Therefore, the cells were carried through eleven days and their VKOR activity followed. FIG. 3 shows that the level of mRNA for gi:13124769 mRNA decreased rapidly to about 20% of normal while VKOR activity decreased continuously during this time period. This reduction in activity is not a general effect of the siRNA or the result of cell death because the level of VKD carboxylase activity and lamin A/C mRNA remained constant. Furthermore, the level of gi:132124769 mRNA is four fold lower in HT-29 cells, which have low VKOR activity, than in A549 cells that exhibit high VKOR activity. These data indicate that gi:13124769 corresponds to the VKOR gene.

### Example 10 Identification of Gene Encoding VKOR

The gene, IMAGE 3455200 (gi:13124769, SEQ ID NO: 8), identified herein to encode VKOR, maps to human chromosome 16p11.2, mouse chromosome 7F3, and rat chromosome 1:180.8 Mb. There are 338 cDNA clones in the NCBI database representing seven different splicing patterns (NCBI AceView program). These are composed of all or part of two to four exons. Among these, the most prevalent isoform, mGC11276, has three exons and is expressed at high levels in lung and liver cells. This three exon transcript (SEQ ID NO: 9) encodes a predicted protein of 163 amino acids with a mass of 18.2 kDa (SEQ ID NO: 10). It is a putative N-myristylated endoplasmic reticulum protein with one to three transmembrane domains, depending upon the program used for prediction. It has seven cysteine residues, which is consistent with observations that the enzymatic activity is dependent upon thiol reagents (Thijssen et al. (1994) “Microsomal lipoamide reductase provides vitamin K epoxide reductase with reducing equivalents” Biochem J 297:277-80). Five of the seven cysteines are conserved among human, mice, rat, zebrafish, Xenopus and Anopheles.

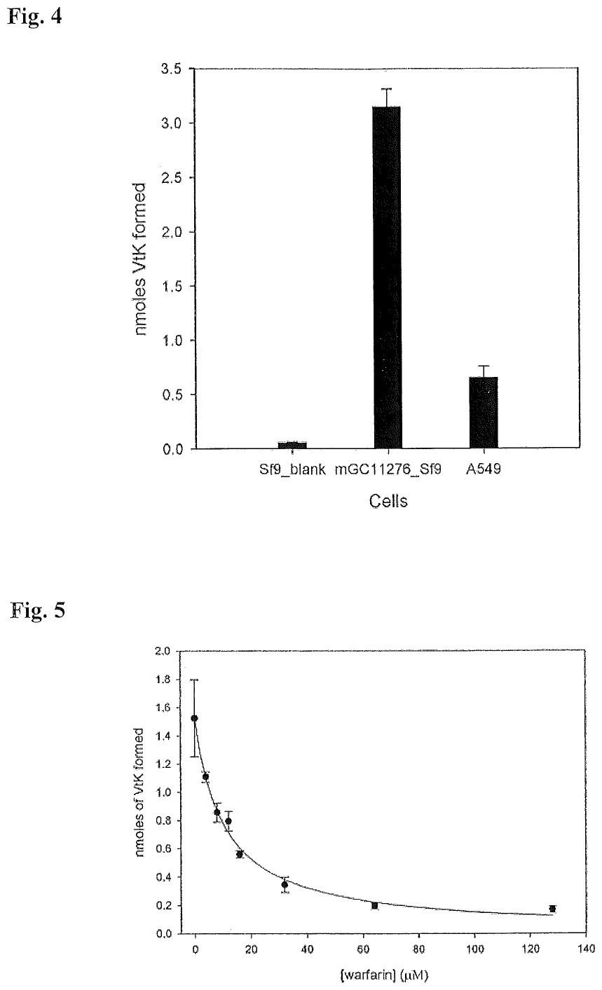

To confirm that the VKOR gene had been identified, the most prevalent form of the enzyme (three exons) was expressed in Spodoptera frugiperda, Sf9 cells. Sf9 cells have no measurable VKOR activity but exhibit warfarin sensitive activity when transfected with mGC11276 cDNA (FIG. 4). VKOR activity is observed from constructs with an epitope tag at either their amino or carboxyl terminus. This tag should assist in the purification of VKOR.

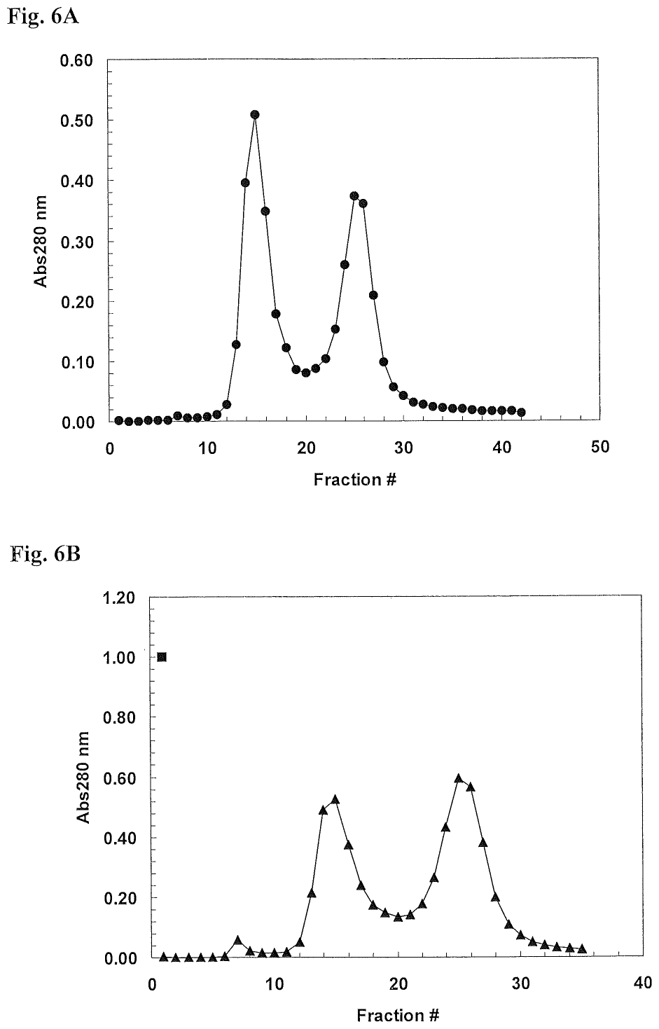

VKOR should exhibit warfarin sensitivity, therefore microsomes were made from Sf9 cells expressing VKOR and tested for warfarin sensitivity. The VKOR activity is warfarin-sensitive (FIG. 5).

In summary, the present invention provides the first example of using siRNA in mammalian cells to identify an unknown gene. The identity of the VKOR gene was confirmed by its expression in insect cells. The VKOR gene encodes several isoforms. It will be important to characterize the activity and expression pattern of each isoform. Millions of people world-wide utilize warfarin to inhibit coagulation; therefore it is important to further characterize VKOR as it can lead to more accurate dosing or design of safer, more effective, anti-coagulants.

### Example 11 Studies on Carboxylation of Factor X

Post translational modification of glutamic acid to gamma carboxy glutamic acid is required for the activity of a number of proteins, most of them related to coagulation. Of these, several have become useful tools for treating various bleeding disorders. For example, recombinant human factor IX now accounts for most of the factor IX used for treating hemophilia B patients. In addition factor Vila is widely used for treating patients with auto-antibodies (inhibitors) to either factor IX or factor VIII and for bleeding that results from general trauma. Another Gla protein, activated protein C, is used for the treatment of sepsis. These vitamin K dependent proteins can be produced in cell culture utilizing cells such as Chinese hamster ovary (CHO), baby hamster kidney cells (BHK) and human embryo kidney cells (HEK 293). A common problem for all of these cell lines is that, if significant overproduction is achieved, then a significant fraction of the recombinant protein produced is under-carboxylated. Originally it was thought that the limiting factor in carboxylation was the vitamin K dependent gamma glutamyl carboxylase. However, after its purification and cloning, it was reported that co-expression of factor IX and carboxylase failed to improve the degree of carboxylation of factor IX in a CHO cell line over-expressing human factor IX. The percentage of carboxylated factor X in the HEK 293 cell line can be increased by reducing the affinity of the factor X's propeptide. However, if the level of expression of factor X bearing the prothrombin propeptide is sufficiently high, the level of expression still exceeds the ability of the cell to achieve complete post-translational modification. The present study demonstrates that co-expressing vitamin K epoxide reductase in a cell line over-expressing factor X (with prothrombin propeptide) to the extent that only about 50% of the factor X is carboxylated, results in its near complete carboxylation.

Materials. All restriction enzymes were from New England Biolabs. Pfu DNA polymerase was obtained from Stratagene. Lipofectamine, hygromycin B and pcDNA3.1/Hygro vector were from Invitrogen. Trypsin-EDTA, fetal bovine serum and Dulbecco's phosphate buffered saline were from Sigma. Antibiotic-antimycotic, G418 (Geneticin) and DMEM F-12 were from GIBCO. Puromycin and the pIRESpuro3 vector were from BD Biosciences. Human factor X was from Enzyme Research Laboratories. Goat anti-human factor X (affinity-purified IgG) and rabbit anti-human factor X (IgG-peroxidase conjugate) were from Affinity Biologicals Corporation. Peroxidase-conjugated AffiniPure rabbit anti-goat IgG was from Jackson ImmunoResearch Laboratories INC. Q-Sepharose™ Fast Flow was obtained from Amersham Pharmacia Biotech. The calcium-dependent monoclonal human FX antibody [MoAb, 4G3] was obtained from Dr. Harold James, University of Texas, Tyler, TX. Bio-Scale CHT5-I Hydroxyapatite was from Bio-Rad Laboratories.

Construction of mammalian cell expression vector containing VKOR. Two primers were designed to amplify the VKOR cDNA. Primer1: 5′-CCGGAATTCGCCGCCACCATGGGCAGCACCTGGGGGAGCCCTGGCTGGGT GCGG (SEQ ID NO:35) introduced a Kozak sequence (underlined) and a 5′ Eco R I site. Primer2: 5′-CGGGCGGCCGCTCAGTGCCTCTTAGCCTTGCC (SEQ ID NO:36) introduced a NotI site at the 3′ terminus of the cDNA. After PCR amplification and digestion with EcoRI and NotI, the PCR product was inserted into pIRESpuro3, which has a CMV virus major immediate early promoter/enhancer and confers puromycin resistance upon the transformed cells.

Construction of mammalian cell expression vector containing HGC. Two primers were designed to amplify HGC cDNA. Primer3: 5′-CGCGGATCCGCCGCCACCATGGCGGTGTCTGCCGGGTCCGCGCGGACCTC GCCC (SEQ ID NO:37) introduced a Bam H1 site and a Kozak sequence (underlined) at the 5′ terminus and Primer4: 5′-CGGGCGGCCGCTCAGAACTCTGAGTGGACAGGATCAGGATTTGACTC (SEQ ID NO:38) introduced a NotI site at the 3′ terminus. After digestion with BamHI and NotI, the PCR product was inserted into pcDNA3.1/Hygro, which has a CMV promoter and confers hygromycin resistance upon the transformed cell.

Stable cell lines expressing Human VKOR. A cell line expressing mutated factor X (HEK293-FXI16L) that produces factor X (half of which is fully carboxylated) at about 10-12 mg per liter was used. HEK293-FXI16L was prepared as described (Camire, 2000) and was selected with the neomycin analogue, G418. HEK293-FXI16L was transfected with the plasmid pIRESpuro3-VKOR using lipofectin (Invitrogen) according to the manufacturer's protocol. Selection was done with 450 μl/ml G418 and 1.75 μl/ml puromycin. Resistant colonies were picked and screened for VKOR activity. The colony with the highest VKOR activity was selected for further analysis.

Stable cell lines expressing Human GGCX. HEK293-FXI16L was transfected, using lipofectin, with the Plasmid pcDNA3.1/Hygro-HGGCX. Transformed colonies were selected with 300 μg/ml of hygromycin and 450 μg/ml of G418 and 18 clones were selected for assay of GGCX activity with the small peptide substrate FLEEL (SEQ ID NO:39). The colony with the highest GGCX activity was selected for further studies.

Stable cell lines co-expressing Human VKOR and HGC. To obtain a HEK293-FXI16L cell line over-expressing both VKOR and GGCX, HEK293-FXI16L-VKOR was transfected with the plasmid pcDNA3.1/Hygro-HGGCX and 18 resistant colonies were selected for analysis. HEK293-FXI16L-HGGCX was also transfected with HEK293-FXI16L-VKOR and from this selection, only one resistant colony was obtained. HEK293-FXI16L was transfected with both pIRESpuro3-VKOR and pcDNA3.1/Hygro-HGC, yielding 10 resistant colonies. The 29 isolated colonies were then assayed for both VKOR and GGCX activity. The clone with the highest levels of both activities was selected for further analysis.

Level of FXI16L production by each cell line. For the sandwich ELISA antibody assay, goat anti-human Factor X (Affinity-Purified IgG) IgG-Peroxidase Conjugate was used as the capture antibody and rabbit anti-human Factor X was used as the detecting antibody. P-OD was used as the substrate for color development. Human factor X was used to make a standard curve. HEK293-FXI16L, HEK293-FXI16L-VKOR, HEK293-FXI16L-HGGCX, and HEK293-FXI16L-VKOR-HGGCX were grown in T25 flasks until they were confluent, then the medium was replaced with serum-free medium containing vitamin K1. The serum-free medium was changed at 12 hours and after 24 hours the conditioned medium was collected and analyzed for FXI16L expression.

Expression of FXI16L from each cell line in roller bottles. The 4 stable cell lines, HEK293-FXI16L, HEK293-FXI16L-VKOR, HEK293-FXI16L-HGGCX, and HEK293-FXI16L-VKOR-HGGCX, were grown in T-225 flasks to confluency and transferred into roller bottles. At 24 and 36 hours the medium was replaced with serum-free medium containing Vitamin K1. The medium was collected from each cell line every 24 hours until a total of three liters was obtained.

Purification of FXI16L from each cell line. The conditioned medium was thawed and passed over a 0.45 μm HVLP filter. EDTA was then added to 5 mM and 0.25 ml of a 100× stock protease inhibitor cocktail was added per liter of conditioned medium. The conditioned media was loaded on a Q-Sepharose™ Fast Flow column equilibrated with 20 mM Tris (pH 7.2)/60 mM NaCl/5 mM EDTA and the column was washed with the same buffer until the baseline was steady. 20 mM Tris (pH 7.2)/700 mM NaCl was used to elute FXI16L from the column. The protein containing fractions were pooled and dialyzed into 8 mM Tris(pH 7.4)/60 mM NaCl. Each sample was made 2 mM CaCl₂) and applied to an immunoaffinity (4G3) column that had been equilibrated with 8 mM Tris(pH 7.4)/60 mM NaCl/2 mM CaCl₂). After washing with the same buffer, eluted factor X was eluted with a linear gradient of 0-8 mM EDTA in the same buffer. The fractions containing protein were pooled and dialyzed overnight into 1 mM Na₂HPO₄/NaH₂PO₄ (pH 6.8). The dialyzed samples were applied to a Bio-Scale CHT5-1 hydroxyapatite column pre-equilibrated with the starting buffer. A linear gradient of 1 to 400 mM Na₂HPO₄/NaH₂PO₄ (pH 6.8) was used to separate carboxylated and non-carboxylated factor X.

Western blotting of sample post Q-sepharose and SDS-PAGE of sample post 4G3. After purification by using Q-Sepharose™ Fast Flow, fractions from four cell lines (HEK293-FXI16L, HEK293-FXI16L-VKOR, HEK293-FXI16L-HGC, and HEK293-VKOR-HGC) were identified by Western blotting. Goat anti-human factor X (Affinity-Purified IgG) was used as first antibody, peroxidase-conjugated affinipure rabbit anti-goat IgG was used as second antibody and ECL substrates were used for developing. After purification by affinity antibody chromatography, some samples were checked for purity.

Analysis of mRNA expression levels for VKOR, HGC and FXI16L among each cell line using real-time Q-PCR. A total of 1×10⁶ cells for each cell line (HEK293-FXI16L, HEK293-FXI16L-VKOR, HEK293-FXI16L-HGC and HEK293-FXI16L-VKOR-HGC) were seeded in a 12 well plate. Total RNA was extracted from each cell line.

VKOR & HGC activity for each cell line (HEK293-FXI16L, HEK293-FXI16L-VKOR, HEK293-FXI16L-HGC and HEK293-FXI16L-VKOR-HGC). pIRESpuro3-VKOR was transfected into HEK293-FXI16L and selected with 1.75 μg/ml puromycin and 450 μg/ml G418. Eighteen single clones were screened for VKOR activity. A single clone that contained a very high level of VKOR activity was kept as a stable cell line, HEK293-FXI16L-VKOR. After pcDNA3.1/Hygro-HGC was transfected into HEK293-FXI16L, transfectants can be selected at 300 μg/ml hygromycin and 450 μg/ml G418. A total of 18 single clones were screened for HGC activity. A single clone that contained a very high level of HGC activity was kept as a stable cell line, HEK293-FXI16L-HGC.

Three methods were used to make the stable cell line that contains a high level of both VKOR and HGC activity. A total of 29 single clones were screened for VKOR and HGC activity. A single clone that contained a high level of both VKOR and HGC activity was kept as a stable cell line HEK293-FXI16L-VKOR-HGC.

FXI16L production in each of the cell line. HEK293-FXI16L-VKOR, HEK293-FXI16L-HGC and HEK293-FXI16L-VKOR-HGC all expressed FXI16L at levels at least as high as the host cell. This experiment was done for comparative purposes in 25 ml T-flasks and the levels of expression were lower than when the protein was prepared in roller bottles. These experiments show that selecting cells over-producing carboxylase or VKOR did not result in loss of factor X expression

Three liters of medium were collected from cells grown in roller bottles and the factor X from each cell line was purified by Q-sepharose and factor X antibody affinity chromatography as described.

Analyzing carboxylation ratio alteration of rFXI16L among each cell line by using hydroxyapatite chromatography. After being dialyzed to 1 mM Na₂HPO₄/NaH₂PO₄ (pH 6.8), fractions post 4G3 were applied to a Bio-Scale CHT5-1 Hydroxyapatite column. A linear gradient of 0-100% of 400 mM Na₂HPO₄/NaH₂PO₄ (pH 6.8) was used to elute column. A total of two pools can be obtained from each sample. The first pool is composed of uncarboxylated human FXI16L, the second pool is composed of fully γ-carboxylated human FXI16L. For each cell line, the amount of fully γ-carboxylated human FXI16L is divided by total amount of human FXI16L, carboxylated to obtain a ratio. The carboxylated ratio for host cell line HEK293-FXI16L is 52% [4.5 mg/(4.13 mg+4.5 mg)=52%]. The carboxylated ratio for the other three cell lines (HEK293-FXI16L-VKOR, HEK293-FXI16L-HGC and HEK293-FXI16L-VKOR-HGC) is 92% [10.5 mg/(0.9 mg+10.5 mg)=92%], 57% [6.4 mg/(4.78 mg+6.4 mg)=57%] and ˜100% [2.4 mg/2.4 mg=100%], respectively.

The big difference in carboxylation ratios between cell lines HEK293-FXI16L and HEK293-FXI16L-VKOR indicates that VKOR improves the γ-carboxylation reaction in vivo dramatically. The smaller difference in carboxylation ratios between cell lines HEK293-FXI16L and HEK293-FXI16L-HGC indicates that although HGC catalyzes the carboxylation reaction, HGC is not the limiting factor of the carboxylation reaction in vivo, and it can only improves the carboxylation reaction in vivo a little. A carboxylation ratio of almost 100% in the cell line HEK293-FXI16L-VKOR-HGC indicates that VKOR can be the limiting factor of the carboxylation reaction in vivo. VKOR not only reduces vitamin K epoxide (KO) to vitamin K, but it also reduces vitamin K to reduced vitamin K (KH₂). Without the second function, which can reduce K to KH₂, vitamin K can not be reused in the carboxylation system in vivo.

In summary, this study demonstrates that a nucleic acid encoding vitamin K epoxide reductase (VKOR), when transfected into cells that have been transfected with and are producing a vitamin K dependent protein, such as factor X, results in the production of a vitamin K dependent protein with increased carboxylation, thereby increasing the amount of active vitamin K dependent protein in the cell.

To do these experiments, a human embryo kidney (HEK) cell line expressing about 12-14 mg per liter of a mutant factor X (with a prothrombin propeptide) was used. This factor X had been modified by replacing its propeptide with the propeptide of prothrombin (Camire et al. “Enhanced gamma-carboxylation of recombinant factor X using a chimeric construct containing the prothrombin propeptide” Biochemistry 39(46):14322-9 (2000)) and was over-producing coagulation factor X to such a great extent that only ˜50% of the factor X was carboxylated.

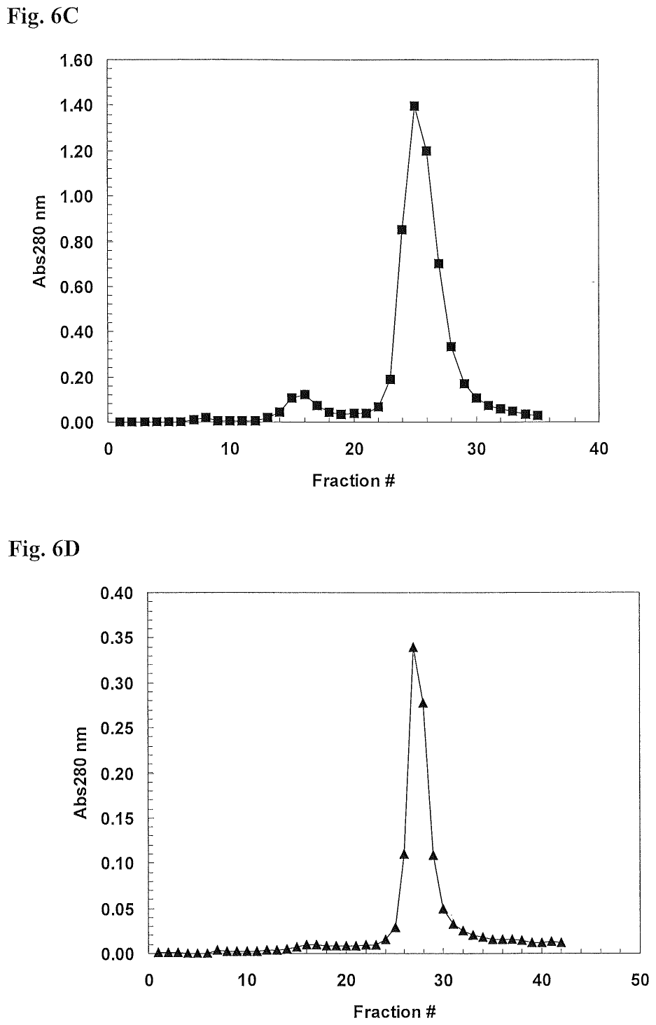

This cell line making about 12-14 mg per liter of factor X was used for the starting and control cells. At this level of expression, the HEK cells could not completely carboxylate the factor X, even with the prothrombin propeptide instead of the normal factor X propeptide. The HEK 293 cells expressing factor X at about 12-14 mg per liter were transfected with 1) vitamin K epoxide reductase (VKOR), 2) vitamin K gamma glutamyl carboxylase, or 3) both vitamin K epoxide reductase and vitamin K gamma glutamyl carboxylase (VKGC). Several cell lines were selected that were shown to produce a large amount of carboxylase, VKOR or both VKOR and carboxylase. In each of these selected cell lines, the level of expression of factor X was at least as high as the starting cell line (within experimental limits). The results of these experiments are shown in FIGS. 6A-D. The comparison in all cases is with the original factor X expressing cell line, which is expressing factor X that is about 50% carboxylated.

Three liters of media were collected from each of the experimental cell lines and the factor X was purified over QAE sephadex, a factor X antibody column and finally a hydroxylapatite column. The figures shown are for the final hydroxylapatite columns. It has previously been shown that the first peak is uncarboxylated factor X and the second peak is fully carboxylated factor X (Camire et al.). FIG. 6A shows results of carboxylation of factor X in the original cell line without exogenous VKOR or VKGC. The second peak (centered around fraction 26) is the fully carboxylated peak. By area, 52% of factor X is fully carboxylated. FIG. 6B shows that adding carboxylase alone to the cell line expressing factor X did not significantly increase the percentage of carboxylated factor X. The extent of full carboxylation increases marginally to 57% fully carboxylated. In this case the fully carboxylated peak is centered around fraction 25. FIG. 6C shows that cells transfected with VKOR alone exhibited dramatically increased levels of fully carboxylated factor X. In this case the fully carboxylated peak (centered around fraction 26) and the extent of full carboxylation is increased to 92% of the total factor X made. FIG. 6D shows that when cells are transfected with both VKOR and VKGC, 100% of the factor X is fully carboxylated. In this situation, expression of the VKOR gene is the main determinant of complete carboxylation of a vitamin K dependent protein. In other situations where the turnover of the substrate is slower, i.e., when the propeptide binds much tighter than the factor X with the prothrombin propeptide and overexpression of the factor X is very high, it is likely that expression of the carboxylase gene will also be limiting. These results can be extended to all vitamin K dependent proteins, in addition to factor X.

These results demonstrate that VKOR (and probably VKGC) facilitates the production of fully carboxylated vitamin K dependent proteins. This provides a mechanism to increase the efficiency of producing fully active, modified proteins.

The foregoing is illustrative of the present invention, and is not to be construed as limiting thereof. The invention is defined by the following claims, with equivalents of the claims to be included therein.

All publications, patent applications, patents, patent publications and other references cited herein are incorporated by reference in their entireties for the teachings relevant to the sentence and/or paragraph in which the reference is presented.

TABLE 1
Five SNPs examined in VKOR gene

| SNPs | position | AA change | Heterozygous ratio |
|---|---|---|---|
| vk563 | 5′-UTR | N/A | 1/58 |
| G > A | | | |
| (SEQ ID NO: 15) | | | |
| vk2581 G > C | Intron2 | N/A | 17/58 |
| (SEQ ID NO: 12) | | | |
| vk3294 T > C | Intron2 | N/A | 25/58 |
| (SEQ ID NO: 13) | | | |
| vk4501 C > T | Exon3 | Leu120Leu | 1/58 |
| (SEQ ID NO: 16 | | | |
| vk4769 G > A | 3′-UTR | N/A | 19/58 |
| (SEQ ID NO: 14 | | | |

TABLE 2

| | VIC | FAM | | |
|---|---|---|---|---|
| | Probe | Probe | Forward | Reverse |
| SNPs | Sequence | Sequence | Primer | Primer |
| vk2581 | TCAT | TCAT | GGTG | CCTG |
| G > C | CACG | CACC | ATCC | TTAG |
| | GAGC | GAGC | ACAC | TTAC |
| | GTC | GTC | AGCT | CTCC |
| | (SEQ | (SEQ | GACA | CCAC |
| | ID | ID | (SEQ | ATC |
| | NO: | NO: | ID | (SEQ |
| | 17) | 18) | NO: | ID |
| | | | 19) | NO: |
| | | | | 20) |
| vk3294 | CCAG | CCAG | GCTC | GCCA |
| T > C | GACC | GACC | CAGA | AGTC |
| | ATGG | GTGG | GAAG | TGAA |
| | TGC | TGC | GCAT | CCAT |
| | (SEQ | (SEQ | CACT | GTGT |
| | ID | ID | (SEQ | CA |
| | NO: | NO: | ID | (SEQ |
| | 21) | 22) | NO: | ID |
| | | | 23) | NO: |
| | | | | 24) |
| vk4769 | ATAC | CATA | GTCC | GTGT |
| G > A | CCGC | CCCA | CTAG | GGCA |
| | ACAT | CACA | AAGG | CATT |
| | GAC | TGAC | CCCT | TGGT |
| | (SEQ | (SEQ | AGAT | CCAT |
| | ID | ID | GT | T |
| | NO: | NO: | (SEQ | (SEQ |
| | 25) | 26) | ID | ID |
| | | | NO: | NO: |
| | | | 27) | 28) |

That which is claimed is:

1. A host cell comprising a recombinant nucleic acid coding for a vitamin K epoxide reductase (VKOR) or a functionally active derivative thereof, and a recombinant nucleic acid coding for a vitamin K dependent protein or a functionally active derivative thereof, wherein both the recombinant VKOR and the recombinant Vitamin K dependent protein are expressed in said host cell.


SEQUENCE LISTING (39 sequences)

SEQ ID NO: 1 - DNA, 21 bp, synthetic construct
  source 1..21: mol_type = other DNA
tccaacagcatattcggttgc

SEQ ID NO: 2 - DNA, 21 bp, synthetic construct
  source 1..21: mol_type = other DNA
ttcttggaccttccggaaact

SEQ ID NO: 3 - DNA, 19 bp, synthetic construct
  source 1..19: mol_type = other DNA
gaaggtgaaggtcggagtc

SEQ ID NO: 4 - DNA, 20 bp, synthetic construct
  source 1..20: mol_type = other DNA
gaagatggtgatgggatttc

SEQ ID NO: 5 - DNA, 21 bp, synthetic construct
  source 1..21: mol_type = other DNA
ctaggtgaggccaagaagcaa

SEQ ID NO: 6 - DNA, 21 bp, synthetic construct
  source 1..21: mol_type = other DNA
ctgttcctctcagcagactgc

SEQ ID NO: 7 - protein, 12 aa, synthetic construct
EDQVDPRLIDGK

SEQ ID NO: 8 - DNA, 3915 bp, Homo sapiens
  source 1..3915: mol_type = genomic DNA
ggttttctccgcgggcgcctcgggcggaacctggagataatgggcagcacctgggggagccctggctgggtgcggctcgctctttgcctgacgggcttagtgctctcgctctacgcgctgcacgtgaaggcggcgcgcgcccgggaccgggattaccgcgcgctctgcgacgtgggcaccgccatcagctgttcgcgcgtcttctcctccaggtgtgcacgggagtgggaggcgtggggcctcggagcagggcggccaggatgccagatgattattctggagtctgggatcggtgtgcccggggaacggacacggggctggactgctcgcggggtcgttgcacaggggctgagctacccagcgatactggtgttcgaaataagagtgcgaggcaagggaccagacagtgctggggactgggattattccggggactcgcacgtgaattggatgccaaggaataacggtgaccaggaaaggcggggaggcaggatggcggtagagattgacgatggtctcaaggacggcgcgcaggtgaaggggggtgttggcgatggctgcgcccaggaacaaggtggcccggtctggctgtgcgtgatggccaggcgttagcataatgacggaatacagaggaggcgagtgagtggccagggagctggagattctggggtccagggcaaagataatctgcccccgactcccagtctctgatgcaaaaccgagtgaaccgttataccagccttgccattttaagaattacttaagggccgggcgcggtggcccactcctgtaatcccagcactttgggaggccgaggcggatggatcacttgaagtcaggagttgaccagcctggccaacatggtgaaagcctgtctctaccaaaaatagaaaaattaatcgggcgctatggcgggtgccttaatcccagctactcgggggggctaaggcaggagaatcgcttgaacccgggaggcggaggtttcagtgagccgagatcgcgccactgcactccagcctgggccagagtgagactccgtctcaaaaaaaaaaaaaaaaaaaaaaaaaaagagacttacttaaggtctaagatgaaaagcagggcctacggagtagccacgtccgggcctggtctggggagaggggaggatagggtcagtgacatggaatcctgacgtggccaaaggtgcccggtgccaggagatcatcgacccttggactaggatgggaggtcggggaacagaggatagcccaggtggcttcttggaaatcacctttctcgggcagggtccaaggcactgggttgacagtcctaacctggttccaccccaccccacccctctgccaggtggggcaggggtttcgggctggtggagcatgtgctgggacaggacagcatcctcaatcaatccaacagcatattcggttgcatcttctacacactacagctattgttaggtgagtggctccgccccctccctgcccgccccgccccgcccctcatcccccttggtcagctcagccccactccatgcaatcttggtgatccacacagctgacagccagctagctgctcatcacggagcgtcctgcgggtggggatgtggggaggtaactaacaggagtcttttaattggtttaagtactgttagaggctgaagggcccttaaagacatcctaggtccccaggttttttgtttgttgttgttttgagacagggtctggctctgttgcccaaagtgaggtctaggatgcccttagtgtgcactggcgtgatctcagttcatggcaacctctgcctccctgcccaagggatcctcccaccttagcctcccaagcagctggaatcacaggcgtgcaccactatgcccagctaatttttgtttttgtttttttttggtagagatggtgtctcgccatgttgcccaggctggtctcaagcaatctgtctgcctcagcctcccaaagtgctggggggattacaggcgtgagctaccatgccccaccaacaccccagttttgtggaaaagatgccgaaattcctttttaaggagaagctgagcatgagctatcttttgtctcatttagtgctcagcaggaaaatttgtatctagtcccataagaacagagagaggaaccaagggagtggaagacgatggcgccccaggccttgctgatgccatatgccggagatgagactatccattaccacccttcccagcaggctcccacgctccctttgagtcacccttcccagctccagagaaggcatcactgagggaggcccagcaccatggtcctggctgacacatggttcagacttggccgatttatttaagaaattttattgctcagaactttccctccctgggcaatggcaagagcttcagagaccagtcccttggaggggacctgttgaagccttctttttttttttttttaagaaataatcttgctctgttgcccaggctggagtgcagtggcacaatcatagctcactgtaacctggctcaagcgatcctcctgagtagctaggactataggcatgtcactgcacccagctaattttttttttttttttttttttttttttgcgacatagtctcgctctgtcaccaggctggagtgcagtggcacgatcttggctcactgcaacctctgcctcccgggttcaagcaattttcctgcctcagcctcctgagtagctgggactacaggcgcgtgtcaccacgcccagctaatttttgtatttttagtggagacagggtttcaccatgttggctaggatggtctcaatctcttgacctggtgatccatccgccttggcctcccaaagtgctaggattacaggcgtgagtcaacctcaccgggcattttttttttgagacgaagtcttgctcttgctgcccaagctggaatgtggtggcatgatctcggctcactgcaacctccacctcctaggttcaagcgattctccaccttagcctccccagcagctgggattacaggtgcccatcaacacacccggctaatttttgtatttttattagagatggggttttgccatgttggccaggctgctctcgaactcctaacctcaggtgatccacccccattggcctcccaaaatactgggattacaggcatgagccaccgtgcccagctgaatttctaaatttttgatagagatcgggtctttctatgttgcccaagctggtcttgaactcctagcctaaagcagtcttcccacctcggcctcccagagtgtttggaatacgtgcgtaagccaccacatctgccctggagcctcttgttttagagacccttcccagcagctcctggcatctaggtagtgcagtgacatcatggagtgttcgggaggtggccagtgcctgaagcccacaccggaccctcttctgccttgcaggttgcctgcggacacgctgggcctctgtcctgatgctgctgagctccctggtgtctctcgctggttctgtctacctggcctggatcctgttcttcgtgctctatgatttctgcattgtttgtatcaccacctatgctatcaacgtgagcctgatgtggctcagtttccggaaggtccaagaaccccagggcaaggctaagaggcactgagccctcaacccaagccaggctgacctcatctgctttgctttggcatgtgagccttgcctaagggggcatatctgggtccctagaaggccctagatgtggggcttctagattaccccctcctcctgccatacccgcacatgacaatggaccaaatgtgccacacgctcgctcttttttacacccagtgcctctgactctgtccccatgggctggtctccaaagctctttccattgcccagggagggaaggttctgagcaataaagtttcttagatcaatca

SEQ ID NO: 9 - DNA, 806 bp, Homo sapiens
  source 1..806: mol_type = other DNA
ggcacgagggttttctccgcgggcgcctcgggcggaacctggagataatgggcagcacctgggggagccctggctgggtgcggctcgctctttgcctgacgggcttagtgctctcgctctacgcgctgcacgtgaaggcggcgcgcgcccgggaccgggattaccgcgcgctctgcgacgtgggcaccgccatcagctgttcgcgcgtcttctcctccaggtggggcaggggtttcgggctggtggagcatgtgctgggacaggacagcatcctcaatcaatccaacagcatattcggttgcatcttctacacactacagctattgttaggttgcctgcggacacgctgggcctctgtcctgatgctgctgagctccctggtgtctctcgctggttctgtctacctggcctggatcctgttcttcgtgctctatgatttctgcattgtttgtatcaccacctatgctatcaacgtgagcctgatgtggctcagtttccggaaggtccaagaaccccagggcaaggctaagaggcactgagccctcaacccaagccaggctgacctcatctgctttgctttggcatgtgagccttgcctaagggggcatatctgggtccctagaaggccctagatgtggggcttctagattaccccctcctcctgccatacccgcacatgacaatggaccaaatgtgccacacgctcgctcttttttacacccagtgcctctgactctgtccccatgggctggtctccaaagctctttccattgcccagggagggaaggttctgagcaataaagttt

SEQ ID NO: 10 - protein, 163 aa, Homo sapiens
MGSTWGSPGWVRLALCLTGLVLSLYALHVKAARARDRDYRALCDVGTAISCSRVFSSRWGRGFGLVEHVLGQDSILNQSNSIFGCIFYTLQLLLGCLRTRWASVLMLLSSLVSLAGSVYLAWILFFVLYDFCIVCITTYAINVSLMWLSFRKVQEPQGKAKRH

SEQ ID NO: 11 - DNA, 5915 bp, Homo sapiens
  source 1..5915: mol_type = genomic DNA
caccatcagatgggacgtctgtgaaggagagacctcatctggcccacagcttggaaaggagagactgactgttgagttgatgcaagctcaggtgttgccaggcgggcgccatgatagtagagaggttaggatactgtcaagggtgtgtgtggccaaaggagtggttctgtgaatgtatgggagaaagggagaccgaccaccaggaagcactggtgaggcaggacccgggaggatgggaggctgcagcccgaatggtgcctgaaatagtttcaggggaaatgcttggttcccgaatcggatcgccgtattcgctggatcccctgatccgctggtctctaggtcccggatgctgcaattcttacaacaggacttggcatagggtaagcgcaaatgctgttaaccacactaacacacttttttttttcttttttttttttgagacagagtctcactctgtcggcctggctggagtgcagtggcacgatctcggctcactgcaacctccggctccccggctcaagcaattctcctgcctcagcctcccgagtagctgggattacaggcatgtgccaccacgcccggctaatttttgtatttttagttgagatggggtttcaccatgttggcgaggctggtcttgaactcctgacctcaggtaatccgccagcctcggcctcccaaagtgctgggattacaagcgtgagccaccgtgcccggccaacagtttttaaatctgtggagacttcatttcccttgatgccttgcagccgcgccgactacaactcccatcatgcctggcagccgctggggccgcgattccgcacgtcccttacccgcttcactagtcccggcattcttcgctgttttcctaactcgcccgcttgactagcgccctggaacagccatttgggtcgtggagtgcgagcacggccggccaatcgccgagtcagagggccaggaggggcgcggccattcgccgcccggcccctgctccgtggctggttttctccgcgggcgcctcgggcggaacctggagataatgggcagcacctgggggagccctggctgggtgcggctcgctctttgcctgacgggcttagtgctctcgctctacgcgctgcacgtgaaggcggcgcgcgcccgggaccgggattaccgcgcgctctgcgacgtgggcaccgccatcagctgttcgcgcgtcttctcctccaggtgtgcacgggagtgggaggcgtggggcctcggagcagggcggccaggatgccagatgattattctggagtctgggatcggtgtgcccggggaacggacacggggctggactgctcgcggggtcgttgcacaggggctgagctacccagcgatactggtgttcgaaataagagtgcgaggcaagggaccagacagtgctggggactgggattattccggggactcgcacgtgaattggatgccaaggaataacggtgaccaggaaaggcggggaggcaggatggcggtagagattgacgatggtctcaaggacggcgcgcaggtgaaggggggtgttggcgatggctgcgcccaggaacaaggtggcccggtctggctgtgcgtgatggccaggcgttagcataatgacggaatacagaggaggcgagtgagtggccagggagctggagattctggggtccagggcaaagataatctgcccccgactcccagtctctgatgcaaaaccgagtgaaccgttataccagccttgccattttaagaattacttaagggccgggcgcggtggcccactcctgtaatcccagcactttgggaggccgaggcggatggatcacttgaagtcaggagttgaccagcctggccaacatggtgaaagcctgtctctaccaaaaatagaaaaattaatcgggcgctatggcgggtgccttaatcccagctactcgggggggctaaggcaggagaatcgcttgaacccgggaggcggaggtttcagtgagccgagatcgcgccactgcactccagcctgggccagagtgagactccgtctcaaaaaaaaaaaaaaaaaaaaaaaaaaagagacttacttaaggtctaagatgaaaagcagggcctacggagtagccacgtccgggcctggtctggggagaggggaggatagggtcagtgacatggaatcctgacgtggccaaaggtgcccggtgccaggagatcatcgacccttggactaggatgggaggtcggggaacagaggatagcccaggtggcttcttggaaatcacctttctcgggcagggtccaaggcactgggttgacagtcctaacctggttccaccccaccccacccctctgccaggtggggcaggggtttcgggctggtggagcatgtgctgggacaggacagcatcctcaatcaatccaacagcatattcggttgcatcttctacacactacagctattgttaggtgagtggctccgccccctccctgcccgccccgccccgcccctcatcccccttggtcagctcagccccactccatgcaatcttggtgatccacacagctgacagccagctagctgctcatcacggagcgtcctgcgggtggggatgtggggaggtaactaacaggagtcttttaattggtttaagtactgttagaggctgaagggcccttaaagacatcctaggtccccaggttttttgtttgttgttgttttgagacagggtctggctctgttgcccaaagtgaggtctaggatgcccttagtgtgcactggcgtgatctcagttcatggcaacctctgcctccctgcccaagggatcctcccaccttagcctcccaagcagctggaatcacaggcgtgcaccactatgcccagctaatttttgtttttgtttttttttggtagagatggtgtctcgccatgttgcccaggctggtctcaagcaatctgtctgcctcagcctcccaaagtgctggggggattacaggcgtgagctaccatgccccaccaacaccccagttttgtggaaaagatgccgaaattcctttttaaggagaagctgagcatgagctatcttttgtctcatttagtgctcagcaggaaaatttgtatctagtcccataagaacagagagaggaaccaagggagtggaagacgatggcgccccaggccttgctgatgccatatgccggagatgagactatccattaccacccttcccagcaggctcccacgctccctttgagtcacccttcccagctccagagaaggcatcactgagggaggcccagcaccatggtcctggctgacacatggttcagacttggccgatttatttaagaaattttattgctcagaactttccctccctgggcaatggcaagagcttcagagaccagtcccttggaggggacctgttgaagccttctttttttttttttttaagaaataatcttgctctgttgcccaggctggagtgcagtggcacaatcatagctcactgtaacctggctcaagcgatcctcctgagtagctaggactataggcatgtcactgcacccagctaattttttttttttttttttttttttttttgcgacatagtctcgctctgtcaccaggctggagtgcagtggcacgatcttggctcactgcaacctctgcctcccgggttcaagcaattttcctgcctcagcctcctgagtagctgggactacaggcgcgtgtcaccacgcccagctaatttttgtatttttagtggagacagggtttcaccatgttggctaggatggtctcaatctcttgacctggtgatccatccgccttggcctcccaaagtgctaggattacaggcgtgagtcaacctcaccgggcattttttttttgagacgaagtcttgctcttgctgcccaagctggaatgtggtggcatgatctcggctcactgcaacctccacctcctaggttcaagcgattctccaccttagcctccccagcagctgggattacaggtgcccatcaacacacccggctaatttttgtatttttattagagatggggttttgccatgttggccaggctgctctcgaactcctaacctcaggtgatccacccccattggcctcccaaaatactgggattacaggcatgagccaccgtgcccagctgaatttctaaatttttgatagagatcgggtctttctatgttgcccaagctggtcttgaactcctagcctaaagcagtcttcccacctcggcctcccagagtgtttggaatacgtgcgtaagccaccacatctgccctggagcctcttgttttagagacccttcccagcagctcctggcatctaggtagtgcagtgacatcatggagtgttcgggaggtggccagtgcctgaagcccacaccggaccctcttctgccttgcaggttgcctgcggacacgctgggcctctgtcctgatgctgctgagctccctggtgtctctcgctggttctgtctacctggcctggatcctgttcttcgtgctctatgatttctgcattgtttgtatcaccacctatgctatcaacgtgagcctgatgtggctcagtttccggaaggtccaagaaccccagggcaaggctaagaggcactgagccctcaacccaagccaggctgacctcatctgctttgctttggcatgtgagccttgcctaagggggcatatctgggtccctagaaggccctagatgtggggcttctagattaccccctcctcctgccatacccgcacatgacaatggaccaaatgtgccacacgctcgctcttttttacacccagtgcctctgactctgtccccatgggctggtctccaaagctctttccattgcccagggagggaaggttctgagcaataaagtttcttagatcaatcagccaagtctgaaccatgtgtctgccatggactgtggtgctgggcctccctcggtgttgccttctctggagctgggaagggtgagtcagagggagagtggagggcctgctgggaagggtggttatgggtagtctcatctccagtgtgtggagtcagcaaggcctggggcaccattggcccccacccccaggaaacaggctggcagctcgctcctgctgcccacaggagccaggcctcctctcctgggaaggctgagcacacacctggaagggcaggctgcccttctggttctgtaaatgcttgctgggaagttcttccttgagtttaactttaacccctccagttgccttatcgaccattccaagccagtattggtagccttggagggtcagggccaggttgtgaaggtttttgttttgcctattatgccctgaccacttacctacatgccaagcactgtttaagaacttgtgttggcagggtgcagtggctcacacctgtaatccctgtactttgggaggccaaggcaggaggatcacttgaggccaggagttccagaccagcctgggcaaaatagtgagacccctgtctctacaaaaaaaaaaaaaaaaaaaaattagccaggcatggtggtgtatgtacctatagtcccaactaatcgggaagctggcgggaagactgcttgagcccagaaggttgaggctgcagtgagccatgatcactgcactccagcctgagcaacagagcaagaccgtctccaaaaaaaaacaaaaaacaaaaaaaaacttgtgttaacgtgttaaactcgtttaatctttacagtgatttatgaggtgggtactattattatccctatcttgatgatagggacagagtggctaattagtatgcctgagatcacacagctactgcaggaggctctcaggatttgaatccacctggtccatctggctccagcatctatatgcttttttttttgttggtttgtttttgagacggac

SEQ ID NO: 12 - DNA, 5915 bp, Homo sapiens
  source 1..5915: mol_type = genomic DNA
caccatcagatgggacgtctgtgaaggagagacctcatctggcccacagcttggaaaggagagactgactgttgagttgatgcaagctcaggtgttgccaggcgggcgccatgatagtagagaggttaggatactgtcaagggtgtgtgtggccaaaggagtggttctgtgaatgtatgggagaaagggagaccgaccaccaggaagcactggtgaggcaggacccgggaggatgggaggctgcagcccgaatggtgcctgaaatagtttcaggggaaatgcttggttcccgaatcggatcgccgtattcgctggatcccctgatccgctggtctctaggtcccggatgctgcaattcttacaacaggacttggcatagggtaagcgcaaatgctgttaaccacactaacacacttttttttttcttttttttttttgagacagagtctcactctgtcggcctggctggagtgcagtggcacgatctcggctcactgcaacctccggctccccggctcaagcaattctcctgcctcagcctcccgagtagctgggattacaggcatgtgccaccacgcccggctaatttttgtatttttagttgagatggggtttcaccatgttggcgaggctggtcttgaactcctgacctcaggtaatccgccagcctcggcctcccaaagtgctgggattacaagcgtgagccaccgtgcccggccaacagtttttaaatctgtggagacttcatttcccttgatgccttgcagccgcgccgactacaactcccatcatgcctggcagccgctggggccgcgattccgcacgtcccttacccgcttcactagtcccggcattcttcgctgttttcctaactcgcccgcttgactagcgccctggaacagccatttgggtcgtggagtgcgagcacggccggccaatcgccgagtcagagggccaggaggggcgcggccattcgccgcccggcccctgctccgtggctggttttctccgcgggcgcctcgggcggaacctggagataatgggcagcacctgggggagccctggctgggtgcggctcgctctttgcctgacgggcttagtgctctcgctctacgcgctgcacgtgaaggcggcgcgcgcccgggaccgggattaccgcgcgctctgcgacgtgggcaccgccatcagctgttcgcgcgtcttctcctccaggtgtgcacgggagtgggaggcgtggggcctcggagcagggcggccaggatgccagatgattattctggagtctgggatcggtgtgcccggggaacggacacggggctggactgctcgcggggtcgttgcacaggggctgagctacccagcgatactggtgttcgaaataagagtgcgaggcaagggaccagacagtgctggggactgggattattccggggactcgcacgtgaattggatgccaaggaataacggtgaccaggaaaggcggggaggcaggatggcggtagagattgacgatggtctcaaggacggcgcgcaggtgaaggggggtgttggcgatggctgcgcccaggaacaaggtggcccggtctggctgtgcgtgatggccaggcgttagcataatgacggaatacagaggaggcgagtgagtggccagggagctggagattctggggtccagggcaaagataatctgcccccgactcccagtctctgatgcaaaaccgagtgaaccgttataccagccttgccattttaagaattacttaagggccgggcgcggtggcccactcctgtaatcccagcactttgggaggccgaggcggatggatcacttgaagtcaggagttgaccagcctggccaacatggtgaaagcctgtctctaccaaaaatagaaaaattaatcgggcgctatggcgggtgccttaatcccagctactcgggggggctaaggcaggagaatcgcttgaacccgggaggcggaggtttcagtgagccgagatcgcgccactgcactccagcctgggccagagtgagactccgtctcaaaaaaaaaaaaaaaaaaaaaaaaaaagagacttacttaaggtctaagatgaaaagcagggcctacggagtagccacgtccgggcctggtctggggagaggggaggatagggtcagtgacatggaatcctgacgtggccaaaggtgcccggtgccaggagatcatcgacccttggactaggatgggaggtcggggaacagaggatagcccaggtggcttcttggaaatcacctttctcgggcagggtccaaggcactgggttgacagtcctaacctggttccaccccaccccacccctctgccaggtggggcaggggtttcgggctggtggagcatgtgctgggacaggacagcatcctcaatcaatccaacagcatattcggttgcatcttctacacactacagctattgttaggtgagtggctccgccccctccctgcccgccccgccccgcccctcatcccccttggtcagctcagccccactccatgcaatcttggtgatccacacagctgacagccagctagctgctcatcaccgagcgtcctgcgggtggggatgtggggaggtaactaacaggagtcttttaattggtttaagtactgttagaggctgaagggcccttaaagacatcctaggtccccaggttttttgtttgttgttgttttgagacagggtctggctctgttgcccaaagtgaggtctaggatgcccttagtgtgcactggcgtgatctcagttcatggcaacctctgcctccctgcccaagggatcctcccaccttagcctcccaagcagctggaatcacaggcgtgcaccactatgcccagctaatttttgtttttgtttttttttggtagagatggtgtctcgccatgttgcccaggctggtctcaagcaatctgtctgcctcagcctcccaaagtgctggggggattacaggcgtgagctaccatgccccaccaacaccccagttttgtggaaaagatgccgaaattcctttttaaggagaagctgagcatgagctatcttttgtctcatttagtgctcagcaggaaaatttgtatctagtcccataagaacagagagaggaaccaagggagtggaagacgatggcgccccaggccttgctgatgccatatgccggagatgagactatccattaccacccttcccagcaggctcccacgctccctttgagtcacccttcccagctccagagaaggcatcactgagggaggcccagcaccatggtcctggctgacacatggttcagacttggccgatttatttaagaaattttattgctcagaactttccctccctgggcaatggcaagagcttcagagaccagtcccttggaggggacctgttgaagccttctttttttttttttttaagaaataatcttgctctgttgcccaggctggagtgcagtggcacaatcatagctcactgtaacctggctcaagcgatcctcctgagtagctaggactataggcatgtcactgcacccagctaattttttttttttttttttttttttttttgcgacatagtctcgctctgtcaccaggctggagtgcagtggcacgatcttggctcactgcaacctctgcctcccgggttcaagcaattttcctgcctcagcctcctgagtagctgggactacaggcgcgtgtcaccacgcccagctaatttttgtatttttagtggagacagggtttcaccatgttggctaggatggtctcaatctcttgacctggtgatccatccgccttggcctcccaaagtgctaggattacaggcgtgagtcaacctcaccgggcattttttttttgagacgaagtcttgctcttgctgcccaagctggaatgtggtggcatgatctcggctcactgcaacctccacctcctaggttcaagcgattctccaccttagcctccccagcagctgggattacaggtgcccatcaacacacccggctaatttttgtatttttattagagatggggttttgccatgttggccaggctgctctcgaactcctaacctcaggtgatccacccccattggcctcccaaaatactgggattacaggcatgagccaccgtgcccagctgaatttctaaatttttgatagagatcgggtctttctatgttgcccaagctggtcttgaactcctagcctaaagcagtcttcccacctcggcctcccagagtgtttggaatacgtgcgtaagccaccacatctgccctggagcctcttgttttagagacccttcccagcagctcctggcatctaggtagtgcagtgacatcatggagtgttcgggaggtggccagtgcctgaagcccacaccggaccctcttctgccttgcaggttgcctgcggacacgctgggcctctgtcctgatgctgctgagctccctggtgtctctcgctggttctgtctacctggcctggatcctgttcttcgtgctctatgatttctgcattgtttgtatcaccacctatgctatcaacgtgagcctgatgtggctcagtttccggaaggtccaagaaccccagggcaaggctaagaggcactgagccctcaacccaagccaggctgacctcatctgctttgctttggcatgtgagccttgcctaagggggcatatctgggtccctagaaggccctagatgtggggcttctagattaccccctcctcctgccatacccgcacatgacaatggaccaaatgtgccacacgctcgctcttttttacacccagtgcctctgactctgtccccatgggctggtctccaaagctctttccattgcccagggagggaaggttctgagcaataaagtttcttagatcaatcagccaagtctgaaccatgtgtctgccatggactgtggtgctgggcctccctcggtgttgccttctctggagctgggaagggtgagtcagagggagagtggagggcctgctgggaagggtggttatgggtagtctcatctccagtgtgtggagtcagcaaggcctggggcaccattggcccccacccccaggaaacaggctggcagctcgctcctgctgcccacaggagccaggcctcctctcctgggaaggctgagcacacacctggaagggcaggctgcccttctggttctgtaaatgcttgctgggaagttcttccttgagtttaactttaacccctccagttgccttatcgaccattccaagccagtattggtagccttggagggtcagggccaggttgtgaaggtttttgttttgcctattatgccctgaccacttacctacatgccaagcactgtttaagaacttgtgttggcagggtgcagtggctcacacctgtaatccctgtactttgggaggccaaggcaggaggatcacttgaggccaggagttccagaccagcctgggcaaaatagtgagacccctgtctctacaaaaaaaaaaaaaaaaaaaaattagccaggcatggtggtgtatgtacctatagtcccaactaatcgggaagctggcgggaagactgcttgagcccagaaggttgaggctgcagtgagccatgatcactgcactccagcctgagcaacagagcaagaccgtctccaaaaaaaaacaaaaaacaaaaaaaaacttgtgttaacgtgttaaactcgtttaatctttacagtgatttatgaggtgggtactattattatccctatcttgatgatagggacagagtggctaattagtatgcctgagatcacacagctactgcaggaggctctcaggatttgaatccacctggtccatctggctccagcatctatatgcttttttttttgttggtttgtttttgagacggac

SEQ ID NO: 13 - DNA, 5915 bp, Homo sapiens
  source 1..5915: mol_type = other DNA
caccatcagatgggacgtctgtgaaggagagacctcatctggcccacagcttggaaaggagagactgactgttgagttgatgcaagctcaggtgttgccaggcgggcgccatgatagtagagaggttaggatactgtcaagggtgtgtgtggccaaaggagtggttctgtgaatgtatgggagaaagggagaccgaccaccaggaagcactggtgaggcaggacccgggaggatgggaggctgcagcccgaatggtgcctgaaatagtttcaggggaaatgcttggttcccgaatcggatcgccgtattcgctggatcccctgatccgctggtctctaggtcccggatgctgcaattcttacaacaggacttggcatagggtaagcgcaaatgctgttaaccacactaacacacttttttttttcttttttttttttgagacagagtctcactctgtcggcctggctggagtgcagtggcacgatctcggctcactgcaacctccggctccccggctcaagcaattctcctgcctcagcctcccgagtagctgggattacaggcatgtgccaccacgcccggctaatttttgtatttttagttgagatggggtttcaccatgttggcgaggctggtcttgaactcctgacctcaggtaatccgccagcctcggcctcccaaagtgctgggattacaagcgtgagccaccgtgcccggccaacagtttttaaatctgtggagacttcatttcccttgatgccttgcagccgcgccgactacaactcccatcatgcctggcagccgctggggccgcgattccgcacgtcccttacccgcttcactagtcccggcattcttcgctgttttcctaactcgcccgcttgactagcgccctggaacagccatttgggtcgtggagtgcgagcacggccggccaatcgccgagtcagagggccaggaggggcgcggccattcgccgcccggcccctgctccgtggctggttttctccgcgggcgcctcgggcggaacctggagataatgggcagcacctgggggagccctggctgggtgcggctcgctctttgcctgacgggcttagtgctctcgctctacgcgctgcacgtgaaggcggcgcgcgcccgggaccgggattaccgcgcgctctgcgacgtgggcaccgccatcagctgttcgcgcgtcttctcctccaggtgtgcacgggagtgggaggcgtggggcctcggagcagggcggccaggatgccagatgattattctggagtctgggatcggtgtgcccggggaacggacacggggctggactgctcgcggggtcgttgcacaggggctgagctacccagcgatactggtgttcgaaataagagtgcgaggcaagggaccagacagtgctggggactgggattattccggggactcgcacgtgaattggatgccaaggaataacggtgaccaggaaaggcggggaggcaggatggcggtagagattgacgatggtctcaaggacggcgcgcaggtgaaggggggtgttggcgatggctgcgcccaggaacaaggtggcccggtctggctgtgcgtgatggccaggcgttagcataatgacggaatacagaggaggcgagtgagtggccagggagctggagattctggggtccagggcaaagataatctgcccccgactcccagtctctgatgcaaaaccgagtgaaccgttataccagccttgccattttaagaattacttaagggccgggcgcggtggcccactcctgtaatcccagcactttgggaggccgaggcggatggatcacttgaagtcaggagttgaccagcctggccaacatggtgaaagcctgtctctaccaaaaatagaaaaattaatcgggcgctatggcgggtgccttaatcccagctactcgggggggctaaggcaggagaatcgcttgaacccgggaggcggaggtttcagtgagccgagatcgcgccactgcactccagcctgggccagagtgagactccgtctcaaaaaaaaaaaaaaaaaaaaaaaaaaagagacttacttaaggtctaagatgaaaagcagggcctacggagtagccacgtccgggcctggtctggggagaggggaggatagggtcagtgacatggaatcctgacgtggccaaaggtgcccggtgccaggagatcatcgacccttggactaggatgggaggtcggggaacagaggatagcccaggtggcttcttggaaatcacctttctcgggcagggtccaaggcactgggttgacagtcctaacctggttccaccccaccccacccctctgccaggtggggcaggggtttcgggctggtggagcatgtgctgggacaggacagcatcctcaatcaatccaacagcatattcggttgcatcttctacacactacagctattgttaggtgagtggctccgccccctccctgcccgccccgccccgcccctcatcccccttggtcagctcagccccactccatgcaatcttggtgatccacacagctgacagccagctagctgctcatcacggagcgtcctgcgggtggggatgtggggaggtaactaacaggagtcttttaattggtttaagtactgttagaggctgaagggcccttaaagacatcctaggtccccaggttttttgtttgttgttgttttgagacagggtctggctctgttgcccaaagtgaggtctaggatgcccttagtgtgcactggcgtgatctcagttcatggcaacctctgcctccctgcccaagggatcctcccaccttagcctcccaagcagctggaatcacaggcgtgcaccactatgcccagctaatttttgtttttgtttttttttggtagagatggtgtctcgccatgttgcccaggctggtctcaagcaatctgtctgcctcagcctcccaaagtgctggggggattacaggcgtgagctaccatgccccaccaacaccccagttttgtggaaaagatgccgaaattcctttttaaggagaagctgagcatgagctatcttttgtctcatttagtgctcagcaggaaaatttgtatctagtcccataagaacagagagaggaaccaagggagtggaagacgatggcgccccaggccttgctgatgccatatgccggagatgagactatccattaccacccttcccagcaggctcccacgctccctttgagtcacccttcccagctccagagaaggcatcactgagggaggcccagcaccacggtcctggctgacacatggttcagacttggccgatttatttaagaaattttattgctcagaactttccctccctgggcaatggcaagagcttcagagaccagtcccttggaggggacctgttgaagccttctttttttttttttttaagaaataatcttgctctgttgcccaggctggagtgcagtggcacaatcatagctcactgtaacctggctcaagcgatcctcctgagtagctaggactataggcatgtcactgcacccagctaattttttttttttttttttttttttttttgcgacatagtctcgctctgtcaccaggctggagtgcagtggcacgatcttggctcactgcaacctctgcctcccgggttcaagcaattttcctgcctcagcctcctgagtagctgggactacaggcgcgtgtcaccacgcccagctaatttttgtatttttagtggagacagggtttcaccatgttggctaggatggtctcaatctcttgacctggtgatccatccgccttggcctcccaaagtgctaggattacaggcgtgagtcaacctcaccgggcattttttttttgagacgaagtcttgctcttgctgcccaagctggaatgtggtggcatgatctcggctcactgcaacctccacctcctaggttcaagcgattctccaccttagcctccccagcagctgggattacaggtgcccatcaacacacccggctaatttttgtatttttattagagatggggttttgccatgttggccaggctgctctcgaactcctaacctcaggtgatccacccccattggcctcccaaaatactgggattacaggcatgagccaccgtgcccagctgaatttctaaatttttgatagagatcgggtctttctatgttgcccaagctggtcttgaactcctagcctaaagcagtcttcccacctcggcctcccagagtgtttggaatacgtgcgtaagccaccacatctgccctggagcctcttgttttagagacccttcccagcagctcctggcatctaggtagtgcagtgacatcatggagtgttcgggaggtggccagtgcctgaagcccacaccggaccctcttctgccttgcaggttgcctgcggacacgctgggcctctgtcctgatgctgctgagctccctggtgtctctcgctggttctgtctacctggcctggatcctgttcttcgtgctctatgatttctgcattgtttgtatcaccacctatgctatcaacgtgagcctgatgtggctcagtttccggaaggtccaagaaccccagggcaaggctaagaggcactgagccctcaacccaagccaggctgacctcatctgctttgctttggcatgtgagccttgcctaagggggcatatctgggtccctagaaggccctagatgtggggcttctagattaccccctcctcctgccatacccgcacatgacaatggaccaaatgtgccacacgctcgctcttttttacacccagtgcctctgactctgtccccatgggctggtctccaaagctctttccattgcccagggagggaaggttctgagcaataaagtttcttagatcaatcagccaagtctgaaccatgtgtctgccatggactgtggtgctgggcctccctcggtgttgccttctctggagctgggaagggtgagtcagagggagagtggagggcctgctgggaagggtggttatgggtagtctcatctccagtgtgtggagtcagcaaggcctggggcaccattggcccccacccccaggaaacaggctggcagctcgctcctgctgcccacaggagccaggcctcctctcctgggaaggctgagcacacacctggaagggcaggctgcccttctggttctgtaaatgcttgctgggaagttcttccttgagtttaactttaacccctccagttgccttatcgaccattccaagccagtattggtagccttggagggtcagggccaggttgtgaaggtttttgttttgcctattatgccctgaccacttacctacatgccaagcactgtttaagaacttgtgttggcagggtgcagtggctcacacctgtaatccctgtactttgggaggccaaggcaggaggatcacttgaggccaggagttccagaccagcctgggcaaaatagtgagacccctgtctctacaaaaaaaaaaaaaaaaaaaaattagccaggcatggtggtgtatgtacctatagtcccaactaatcgggaagctggcgggaagactgcttgagcccagaaggttgaggctgcagtgagccatgatcactgcactccagcctgagcaacagagcaagaccgtctccaaaaaaaaacaaaaaacaaaaaaaaacttgtgttaacgtgttaaactcgtttaatctttacagtgatttatgaggtgggtactattattatccctatcttgatgatagggacagagtggctaattagtatgcctgagatcacacagctactgcaggaggctctcaggatttgaatccacctggtccatctggctccagcatctatatgcttttttttttgttggtttgtttttgagacggac

SEQ ID NO: 14 - DNA, 5915 bp, Homo sapiens
  source 1..5915: mol_type = genomic DNA
caccatcagatgggacgtctgtgaaggagagacctcatctggcccacagcttggaaaggagagactgactgttgagttgatgcaagctcaggtgttgccaggcgggcgccatgatagtagagaggttaggatactgtcaagggtgtgtgtggccaaaggagtggttctgtgaatgtatgggagaaagggagaccgaccaccaggaagcactggtgaggcaggacccgggaggatgggaggctgcagcccgaatggtgcctgaaatagtttcaggggaaatgcttggttcccgaatcggatcgccgtattcgctggatcccctgatccgctggtctctaggtcccggatgctgcaattcttacaacaggacttggcatagggtaagcgcaaatgctgttaaccacactaacacacttttttttttcttttttttttttgagacagagtctcactctgtcggcctggctggagtgcagtggcacgatctcggctcactgcaacctccggctccccggctcaagcaattctcctgcctcagcctcccgagtagctgggattacaggcatgtgccaccacgcccggctaatttttgtatttttagttgagatggggtttcaccatgttggcgaggctggtcttgaactcctgacctcaggtaatccgccagcctcggcctcccaaagtgctgggattacaagcgtgagccaccgtgcccggccaacagtttttaaatctgtggagacttcatttcccttgatgccttgcagccgcgccgactacaactcccatcatgcctggcagccgctggggccgcgattccgcacgtcccttacccgcttcactagtcccggcattcttcgctgttttcctaactcgcccgcttgactagcgccctggaacagccatttgggtcgtggagtgcgagcacggccggccaatcgccgagtcagagggccaggaggggcgcggccattcgccgcccggcccctgctccgtggctggttttctccgcgggcgcctcgggcggaacctggagataatgggcagcacctgggggagccctggctgggtgcggctcgctctttgcctgacgggcttagtgctctcgctctacgcgctgcacgtgaaggcggcgcgcgcccgggaccgggattaccgcgcgctctgcgacgtgggcaccgccatcagctgttcgcgcgtcttctcctccaggtgtgcacgggagtgggaggcgtggggcctcggagcagggcggccaggatgccagatgattattctggagtctgggatcggtgtgcccggggaacggacacggggctggactgctcgcggggtcgttgcacaggggctgagctacccagcgatactggtgttcgaaataagagtgcgaggcaagggaccagacagtgctggggactgggattattccggggactcgcacgtgaattggatgccaaggaataacggtgaccaggaaaggcggggaggcaggatggcggtagagattgacgatggtctcaaggacggcgcgcaggtgaaggggggtgttggcgatggctgcgcccaggaacaaggtggcccggtctggctgtgcgtgatggccaggcgttagcataatgacggaatacagaggaggcgagtgagtggccagggagctggagattctggggtccagggcaaagataatctgcccccgactcccagtctctgatgcaaaaccgagtgaaccgttataccagccttgccattttaagaattacttaagggccgggcgcggtggcccactcctgtaatcccagcactttgggaggccgaggcggatggatcacttgaagtcaggagttgaccagcctggccaacatggtgaaagcctgtctctaccaaaaatagaaaaattaatcgggcgctatggcgggtgccttaatcccagctactcgggggggctaaggcaggagaatcgcttgaacccgggaggcggaggtttcagtgagccgagatcgcgccactgcactccagcctgggccagagtgagactccgtctcaaaaaaaaaaaaaaaaaaaaaaaaaaagagacttacttaaggtctaagatgaaaagcagggcctacggagtagccacgtccgggcctggtctggggagaggggaggatagggtcagtgacatggaatcctgacgtggccaaaggtgcccggtgccaggagatcatcgacccttggactaggatgggaggtcggggaacagaggatagcccaggtggcttcttggaaatcacctttctcgggcagggtccaaggcactgggttgacagtcctaacctggttccaccccaccccacccctctgccaggtggggcaggggtttcgggctggtggagcatgtgctgggacaggacagcatcctcaatcaatccaacagcatattcggttgcatcttctacacactacagctattgttaggtgagtggctccgccccctccctgcccgccccgccccgcccctcatcccccttggtcagctcagccccactccatgcaatcttggtgatccacacagctgacagccagctagctgctcatcacggagcgtcctgcgggtggggatgtggggaggtaactaacaggagtcttttaattggtttaagtactgttagaggctgaagggcccttaaagacatcctaggtccccaggttttttgtttgttgttgttttgagacagggtctggctctgttgcccaaagtgaggtctaggatgcccttagtgtgcactggcgtgatctcagttcatggcaacctctgcctccctgcccaagggatcctcccaccttagcctcccaagcagctggaatcacaggcgtgcaccactatgcccagctaatttttgtttttgtttttttttggtagagatggtgtctcgccatgttgcccaggctggtctcaagcaatctgtctgcctcagcctcccaaagtgctggggggattacaggcgtgagctaccatgccccaccaacaccccagttttgtggaaaagatgccgaaattcctttttaaggagaagctgagcatgagctatcttttgtctcatttagtgctcagcaggaaaatttgtatctagtcccataagaacagagagaggaaccaagggagtggaagacgatggcgccccaggccttgctgatgccatatgccggagatgagactatccattaccacccttcccagcaggctcccacgctccctttgagtcacccttcccagctccagagaaggcatcactgagggaggcccagcaccatggtcctggctgacacatggttcagacttggccgatttatttaagaaattttattgctcagaactttccctccctgggcaatggcaagagcttcagagaccagtcccttggaggggacctgttgaagccttctttttttttttttttaagaaataatcttgctctgttgcccaggctggagtgcagtggcacaatcatagctcactgtaacctggctcaagcgatcctcctgagtagctaggactataggcatgtcactgcacccagctaattttttttttttttttttttttttttttgcgacatagtctcgctctgtcaccaggctggagtgcagtggcacgatcttggctcactgcaacctctgcctcccgggttcaagcaattttcctgcctcagcctcctgagtagctgggactacaggcgcgtgtcaccacgcccagctaatttttgtatttttagtggagacagggtttcaccatgttggctaggatggtctcaatctcttgacctggtgatccatccgccttggcctcccaaagtgctaggattacaggcgtgagtcaacctcaccgggcattttttttttgagacgaagtcttgctcttgctgcccaagctggaatgtggtggcatgatctcggctcactgcaacctccacctcctaggttcaagcgattctccaccttagcctccccagcagctgggattacaggtgcccatcaacacacccggctaatttttgtatttttattagagatggggttttgccatgttggccaggctgctctcgaactcctaacctcaggtgatccacccccattggcctcccaaaatactgggattacaggcatgagccaccgtgcccagctgaatttctaaatttttgatagagatcgggtctttctatgttgcccaagctggtcttgaactcctagcctaaagcagtcttcccacctcggcctcccagagtgtttggaatacgtgcgtaagccaccacatctgccctggagcctcttgttttagagacccttcccagcagctcctggcatctaggtagtgcagtgacatcatggagtgttcgggaggtggccagtgcctgaagcccacaccggaccctcttctgccttgcaggttgcctgcggacacgctgggcctctgtcctgatgctgctgagctccctggtgtctctcgctggttctgtctacctggcctggatcctgttcttcgtgctctatgatttctgcattgtttgtatcaccacctatgctatcaacgtgagcctgatgtggctcagtttccggaaggtccaagaaccccagggcaaggctaagaggcactgagccctcaacccaagccaggctgacctcatctgctttgctttggcatgtgagccttgcctaagggggcatatctgggtccctagaaggccctagatgtggggcttctagattaccccctcctcctgccatacccacacatgacaatggaccaaatgtgccacacgctcgctcttttttacacccagtgcctctgactctgtccccatgggctggtctccaaagctctttccattgcccagggagggaaggttctgagcaataaagtttcttagatcaatcagccaagtctgaaccatgtgtctgccatggactgtggtgctgggcctccctcggtgttgccttctctggagctgggaagggtgagtcagagggagagtggagggcctgctgggaagggtggttatgggtagtctcatctccagtgtgtggagtcagcaaggcctggggcaccattggcccccacccccaggaaacaggctggcagctcgctcctgctgcccacaggagccaggcctcctctcctgggaaggctgagcacacacctggaagggcaggctgcccttctggttctgtaaatgcttgctgggaagttcttccttgagtttaactttaacccctccagttgccttatcgaccattccaagccagtattggtagccttggagggtcagggccaggttgtgaaggtttttgttttgcctattatgccctgaccacttacctacatgccaagcactgtttaagaacttgtgttggcagggtgcagtggctcacacctgtaatccctgtactttgggaggccaaggcaggaggatcacttgaggccaggagttccagaccagcctgggcaaaatagtgagacccctgtctctacaaaaaaaaaaaaaaaaaaaaattagccaggcatggtggtgtatgtacctatagtcccaactaatcgggaagctggcgggaagactgcttgagcccagaaggttgaggctgcagtgagccatgatcactgcactccagcctgagcaacagagcaagaccgtctccaaaaaaaaacaaaaaacaaaaaaaaacttgtgttaacgtgttaaactcgtttaatctttacagtgatttatgaggtgggtactattattatccctatcttgatgatagggacagagtggctaattagtatgcctgagatcacacagctactgcaggaggctctcaggatttgaatccacctggtccatctggctccagcatctatatgcttttttttttgttggtttgtttttgagacggac

SEQ ID NO: 15 - DNA, 5915 bp, Homo sapiens
  source 1..5915: mol_type = genomic DNA
caccatcagatgggacgtctgtgaaggagagacctcatctggcccacagcttggaaaggagagactgactgttgagttgatgcaagctcaggtgttgccaggcgggcgccatgatagtagagaggttaggatactgtcaagggtgtgtgtggccaaaggagtggttctgtgaatgtatgggagaaagggagaccgaccaccaggaagcactggtgaggcaggacccgggaggatgggaggctgcagcccgaatggtgcctgaaatagtttcaggggaaatgcttggttcccgaatcggatcgccgtattcgctggatcccctgatccgctggtctctaggtcccggatgctgcaattcttacaacaggacttggcatagggtaagcgcaaatgctgttaaccacactaacacacttttttttttcttttttttttttgagacagagtctcactctgtcggcctggctggagtgcagtggcacgatctcggctcactgcaacctccggctccccggctcaagcaattctcctgcctcagcctcccgagtagctgggattacagacatgtgccaccacgcccggctaatttttgtatttttagttgagatggggtttcaccatgttggcgaggctggtcttgaactcctgacctcaggtaatccgccagcctcggcctcccaaagtgctgggattacaagcgtgagccaccgtgcccggccaacagtttttaaatctgtggagacttcatttcccttgatgccttgcagccgcgccgactacaactcccatcatgcctggcagccgctggggccgcgattccgcacgtcccttacccgcttcactagtcccggcattcttcgctgttttcctaactcgcccgcttgactagcgccctggaacagccatttgggtcgtggagtgcgagcacggccggccaatcgccgagtcagagggccaggaggggcgcggccattcgccgcccggcccctgctccgtggctggttttctccgcgggcgcctcgggcggaacctggagataatgggcagcacctgggggagccctggctgggtgcggctcgctctttgcctgacgggcttagtgctctcgctctacgcgctgcacgtgaaggcggcgcgcgcccgggaccgggattaccgcgcgctctgcgacgtgggcaccgccatcagctgttcgcgcgtcttctcctccaggtgtgcacgggagtgggaggcgtggggcctcggagcagggcggccaggatgccagatgattattctggagtctgggatcggtgtgcccggggaacggacacggggctggactgctcgcggggtcgttgcacaggggctgagctacccagcgatactggtgttcgaaataagagtgcgaggcaagggaccagacagtgctggggactgggattattccggggactcgcacgtgaattggatgccaaggaataacggtgaccaggaaaggcggggaggcaggatggcggtagagattgacgatggtctcaaggacggcgcgcaggtgaaggggggtgttggcgatggctgcgcccaggaacaaggtggcccggtctggctgtgcgtgatggccaggcgttagcataatgacggaatacagaggaggcgagtgagtggccagggagctggagattctggggtccagggcaaagataatctgcccccgactcccagtctctgatgcaaaaccgagtgaaccgttataccagccttgccattttaagaattacttaagggccgggcgcggtggcccactcctgtaatcccagcactttgggaggccgaggcggatggatcacttgaagtcaggagttgaccagcctggccaacatggtgaaagcctgtctctaccaaaaatagaaaaattaatcgggcgctatggcgggtgccttaatcccagctactcgggggggctaaggcaggagaatcgcttgaacccgggaggcggaggtttcagtgagccgagatcgcgccactgcactccagcctgggccagagtgagactccgtctcaaaaaaaaaaaaaaaaaaaaaaaaaaagagacttacttaaggtctaagatgaaaagcagggcctacggagtagccacgtccgggcctggtctggggagaggggaggatagggtcagtgacatggaatcctgacgtggccaaaggtgcccggtgccaggagatcatcgacccttggactaggatgggaggtcggggaacagaggatagcccaggtggcttcttggaaatcacctttctcgggcagggtccaaggcactgggttgacagtcctaacctggttccaccccaccccacccctctgccaggtggggcaggggtttcgggctggtggagcatgtgctgggacaggacagcatcctcaatcaatccaacagcatattcggttgcatcttctacacactacagctattgttaggtgagtggctccgccccctccctgcccgccccgccccgcccctcatcccccttggtcagctcagccccactccatgcaatcttggtgatccacacagctgacagccagctagctgctcatcacggagcgtcctgcgggtggggatgtggggaggtaactaacaggagtcttttaattggtttaagtactgttagaggctgaagggcccttaaagacatcctaggtccccaggttttttgtttgttgttgttttgagacagggtctggctctgttgcccaaagtgaggtctaggatgcccttagtgtgcactggcgtgatctcagttcatggcaacctctgcctccctgcccaagggatcctcccaccttagcctcccaagcagctggaatcacaggcgtgcaccactatgcccagctaatttttgtttttgtttttttttggtagagatggtgtctcgccatgttgcccaggctggtctcaagcaatctgtctgcctcagcctcccaaagtgctggggggattacaggcgtgagctaccatgccccaccaacaccccagttttgtggaaaagatgccgaaattcctttttaaggagaagctgagcatgagctatcttttgtctcatttagtgctcagcaggaaaatttgtatctagtcccataagaacagagagaggaaccaagggagtggaagacgatggcgccccaggccttgctgatgccatatgccggagatgagactatccattaccacccttcccagcaggctcccacgctccctttgagtcacccttcccagctccagagaaggcatcactgagggaggcccagcaccatggtcctggctgacacatggttcagacttggccgatttatttaagaaattttattgctcagaactttccctccctgggcaatggcaagagcttcagagaccagtcccttggaggggacctgttgaagccttctttttttttttttttaagaaataatcttgctctgttgcccaggctggagtgcagtggcacaatcatagctcactgtaacctggctcaagcgatcctcctgagtagctaggactataggcatgtcactgcacccagctaattttttttttttttttttttttttttttgcgacatagtctcgctctgtcaccaggctggagtgcagtggcacgatcttggctcactgcaacctctgcctcccgggttcaagcaattttcctgcctcagcctcctgagtagctgggactacaggcgcgtgtcaccacgcccagctaatttttgtatttttagtggagacagggtttcaccatgttggctaggatggtctcaatctcttgacctggtgatccatccgccttggcctcccaaagtgctaggattacaggcgtgagtcaacctcaccgggcattttttttttgagacgaagtcttgctcttgctgcccaagctggaatgtggtggcatgatctcggctcactgcaacctccacctcctaggttcaagcgattctccaccttagcctccccagcagctgggattacaggtgcccatcaacacacccggctaatttttgtatttttattagagatggggttttgccatgttggccaggctgctctcgaactcctaacctcaggtgatccacccccattggcctcccaaaatactgggattacaggcatgagccaccgtgcccagctgaatttctaaatttttgatagagatcgggtctttctatgttgcccaagctggtcttgaactcctagcctaaagcagtcttcccacctcggcctcccagagtgtttggaatacgtgcgtaagccaccacatctgccctggagcctcttgttttagagacccttcccagcagctcctggcatctaggtagtgcagtgacatcatggagtgttcgggaggtggccagtgcctgaagcccacaccggaccctcttctgccttgcaggttgcctgcggacacgctgggcctctgtcctgatgctgctgagctccctggtgtctctcgctggttctgtctacctggcctggatcctgttcttcgtgctctatgatttctgcattgtttgtatcaccacctatgctatcaacgtgagcctgatgtggctcagtttccggaaggtccaagaaccccagggcaaggctaagaggcactgagccctcaacccaagccaggctgacctcatctgctttgctttggcatgtgagccttgcctaagggggcatatctgggtccctagaaggccctagatgtggggcttctagattaccccctcctcctgccatacccgcacatgacaatggaccaaatgtgccacacgctcgctcttttttacacccagtgcctctgactctgtccccatgggctggtctccaaagctctttccattgcccagggagggaaggttctgagcaataaagtttcttagatcaatcagccaagtctgaaccatgtgtctgccatggactgtggtgctgggcctccctcggtgttgccttctctggagctgggaagggtgagtcagagggagagtggagggcctgctgggaagggtggttatgggtagtctcatctccagtgtgtggagtcagcaaggcctggggcaccattggcccccacccccaggaaacaggctggcagctcgctcctgctgcccacaggagccaggcctcctctcctgggaaggctgagcacacacctggaagggcaggctgcccttctggttctgtaaatgcttgctgggaagttcttccttgagtttaactttaacccctccagttgccttatcgaccattccaagccagtattggtagccttggagggtcagggccaggttgtgaaggtttttgttttgcctattatgccctgaccacttacctacatgccaagcactgtttaagaacttgtgttggcagggtgcagtggctcacacctgtaatccctgtactttgggaggccaaggcaggaggatcacttgaggccaggagttccagaccagcctgggcaaaatagtgagacccctgtctctacaaaaaaaaaaaaaaaaaaaaattagccaggcatggtggtgtatgtacctatagtcccaactaatcgggaagctggcgggaagactgcttgagcccagaaggttgaggctgcagtgagccatgatcactgcactccagcctgagcaacagagcaagaccgtctccaaaaaaaaacaaaaaacaaaaaaaaacttgtgttaacgtgttaaactcgtttaatctttacagtgatttatgaggtgggtactattattatccctatcttgatgatagggacagagtggctaattagtatgcctgagatcacacagctactgcaggaggctctcaggatttgaatccacctggtccatctggctccagcatctatatgcttttttttttgttggtttgtttttgagacggac

SEQ ID NO: 16 - DNA, 5915 bp, Homo sapiens
  source 1..5915: mol_type = genomic DNA
caccatcagatgggacgtctgtgaaggagagacctcatctggcccacagcttggaaaggagagactgactgttgagttgatgcaagctcaggtgttgccaggcgggcgccatgatagtagagaggttaggatactgtcaagggtgtgtgtggccaaaggagtggttctgtgaatgtatgggagaaagggagaccgaccaccaggaagcactggtgaggcaggacccgggaggatgggaggctgcagcccgaatggtgcctgaaatagtttcaggggaaatgcttggttcccgaatcggatcgccgtattcgctggatcccctgatccgctggtctctaggtcccggatgctgcaattcttacaacaggacttggcatagggtaagcgcaaatgctgttaaccacactaacacacttttttttttcttttttttttttgagacagagtctcactctgtcggcctggctggagtgcagtggcacgatctcggctcactgcaacctccggctccccggctcaagcaattctcctgcctcagcctcccgagtagctgggattacaggcatgtgccaccacgcccggctaatttttgtatttttagttgagatggggtttcaccatgttggcgaggctggtcttgaactcctgacctcaggtaatccgccagcctcggcctcccaaagtgctgggattacaagcgtgagccaccgtgcccggccaacagtttttaaatctgtggagacttcatttcccttgatgccttgcagccgcgccgactacaactcccatcatgcctggcagccgctggggccgcgattccgcacgtcccttacccgcttcactagtcccggcattcttcgctgttttcctaactcgcccgcttgactagcgccctggaacagccatttgggtcgtggagtgcgagcacggccggccaatcgccgagtcagagggccaggaggggcgcggccattcgccgcccggcccctgctccgtggctggttttctccgcgggcgcctcgggcggaacctggagataatgggcagcacctgggggagccctggctgggtgcggctcgctctttgcctgacgggcttagtgctctcgctctacgcgctgcacgtgaaggcggcgcgcgcccgggaccgggattaccgcgcgctctgcgacgtgggcaccgccatcagctgttcgcgcgtcttctcctccaggtgtgcacgggagtgggaggcgtggggcctcggagcagggcggccaggatgccagatgattattctggagtctgggatcggtgtgcccggggaacggacacggggctggactgctcgcggggtcgttgcacaggggctgagctacccagcgatactggtgttcgaaataagagtgcgaggcaagggaccagacagtgctggggactgggattattccggggactcgcacgtgaattggatgccaaggaataacggtgaccaggaaaggcggggaggcaggatggcggtagagattgacgatggtctcaaggacggcgcgcaggtgaaggggggtgttggcgatggctgcgcccaggaacaaggtggcccggtctggctgtgcgtgatggccaggcgttagcataatgacggaatacagaggaggcgagtgagtggccagggagctggagattctggggtccagggcaaagataatctgcccccgactcccagtctctgatgcaaaaccgagtgaaccgttataccagccttgccattttaagaattacttaagggccgggcgcggtggcccactcctgtaatcccagcactttgggaggccgaggcggatggatcacttgaagtcaggagttgaccagcctggccaacatggtgaaagcctgtctctaccaaaaatagaaaaattaatcgggcgctatggcgggtgccttaatcccagctactcgggggggctaaggcaggagaatcgcttgaacccgggaggcggaggtttcagtgagccgagatcgcgccactgcactccagcctgggccagagtgagactccgtctcaaaaaaaaaaaaaaaaaaaaaaaaaaagagacttacttaaggtctaagatgaaaagcagggcctacggagtagccacgtccgggcctggtctggggagaggggaggatagggtcagtgacatggaatcctgacgtggccaaaggtgcccggtgccaggagatcatcgacccttggactaggatgggaggtcggggaacagaggatagcccaggtggcttcttggaaatcacctttctcgggcagggtccaaggcactgggttgacagtcctaacctggttccaccccaccccacccctctgccaggtggggcaggggtttcgggctggtggagcatgtgctgggacaggacagcatcctcaatcaatccaacagcatattcggttgcatcttctacacactacagctattgttaggtgagtggctccgccccctccctgcccgccccgccccgcccctcatcccccttggtcagctcagccccactccatgcaatcttggtgatccacacagctgacagccagctagctgctcatcacggagcgtcctgcgggtggggatgtggggaggtaactaacaggagtcttttaattggtttaagtactgttagaggctgaagggcccttaaagacatcctaggtccccaggttttttgtttgttgttgttttgagacagggtctggctctgttgcccaaagtgaggtctaggatgcccttagtgtgcactggcgtgatctcagttcatggcaacctctgcctccctgcccaagggatcctcccaccttagcctcccaagcagctggaatcacaggcgtgcaccactatgcccagctaatttttgtttttgtttttttttggtagagatggtgtctcgccatgttgcccaggctggtctcaagcaatctgtctgcctcagcctcccaaagtgctggggggattacaggcgtgagctaccatgccccaccaacaccccagttttgtggaaaagatgccgaaattcctttttaaggagaagctgagcatgagctatcttttgtctcatttagtgctcagcaggaaaatttgtatctagtcccataagaacagagagaggaaccaagggagtggaagacgatggcgccccaggccttgctgatgccatatgccggagatgagactatccattaccacccttcccagcaggctcccacgctccctttgagtcacccttcccagctccagagaaggcatcactgagggaggcccagcaccatggtcctggctgacacatggttcagacttggccgatttatttaagaaattttattgctcagaactttccctccctgggcaatggcaagagcttcagagaccagtcccttggaggggacctgttgaagccttctttttttttttttttaagaaataatcttgctctgttgcccaggctggagtgcagtggcacaatcatagctcactgtaacctggctcaagcgatcctcctgagtagctaggactataggcatgtcactgcacccagctaattttttttttttttttttttttttttttgcgacatagtctcgctctgtcaccaggctggagtgcagtggcacgatcttggctcactgcaacctctgcctcccgggttcaagcaattttcctgcctcagcctcctgagtagctgggactacaggcgcgtgtcaccacgcccagctaatttttgtatttttagtggagacagggtttcaccatgttggctaggatggtctcaatctcttgacctggtgatccatccgccttggcctcccaaagtgctaggattacaggcgtgagtcaacctcaccgggcattttttttttgagacgaagtcttgctcttgctgcccaagctggaatgtggtggcatgatctcggctcactgcaacctccacctcctaggttcaagcgattctccaccttagcctccccagcagctgggattacaggtgcccatcaacacacccggctaatttttgtatttttattagagatggggttttgccatgttggccaggctgctctcgaactcctaacctcaggtgatccacccccattggcctcccaaaatactgggattacaggcatgagccaccgtgcccagctgaatttctaaatttttgatagagatcgggtctttctatgttgcccaagctggtcttgaactcctagcctaaagcagtcttcccacctcggcctcccagagtgtttggaatacgtgcgtaagccaccacatctgccctggagcctcttgttttagagacccttcccagcagctcctggcatctaggtagtgcagtgacatcatggagtgttcgggaggtggccagtgcctgaagcccacaccggaccctcttctgccttgcaggttgcctgcggacacgctgggcctctgtcctgatgctgctgagctccctggtgtctctcgctggttctgtctacttggcctggatcctgttcttcgtgctctatgatttctgcattgtttgtatcaccacctatgctatcaacgtgagcctgatgtggctcagtttccggaaggtccaagaaccccagggcaaggctaagaggcactgagccctcaacccaagccaggctgacctcatctgctttgctttggcatgtgagccttgcctaagggggcatatctgggtccctagaaggccctagatgtggggcttctagattaccccctcctcctgccatacccgcacatgacaatggaccaaatgtgccacacgctcgctcttttttacacccagtgcctctgactctgtccccatgggctggtctccaaagctctttccattgcccagggagggaaggttctgagcaataaagtttcttagatcaatcagccaagtctgaaccatgtgtctgccatggactgtggtgctgggcctccctcggtgttgccttctctggagctgggaagggtgagtcagagggagagtggagggcctgctgggaagggtggttatgggtagtctcatctccagtgtgtggagtcagcaaggcctggggcaccattggcccccacccccaggaaacaggctggcagctcgctcctgctgcccacaggagccaggcctcctctcctgggaaggctgagcacacacctggaagggcaggctgcccttctggttctgtaaatgcttgctgggaagttcttccttgagtttaactttaacccctccagttgccttatcgaccattccaagccagtattggtagccttggagggtcagggccaggttgtgaaggtttttgttttgcctattatgccctgaccacttacctacatgccaagcactgtttaagaacttgtgttggcagggtgcagtggctcacacctgtaatccctgtactttgggaggccaaggcaggaggatcacttgaggccaggagttccagaccagcctgggcaaaatagtgagacccctgtctctacaaaaaaaaaaaaaaaaaaaaattagccaggcatggtggtgtatgtacctatagtcccaactaatcgggaagctggcgggaagactgcttgagcccagaaggttgaggctgcagtgagccatgatcactgcactccagcctgagcaacagagcaagaccgtctccaaaaaaaaacaaaaaacaaaaaaaaacttgtgttaacgtgttaaactcgtttaatctttacagtgatttatgaggtgggtactattattatccctatcttgatgatagggacagagtggctaattagtatgcctgagatcacacagctactgcaggaggctctcaggatttgaatccacctggtccatctggctccagcatctatatgcttttttttttgttggtttgtttttgagacggac

SEQ ID NO: 17 - DNA, 15 bp, synthetic construct
  source 1..15: mol_type = other DNA
tcatcacggagcgtc

SEQ ID NO: 18 - DNA, 15 bp, synthetic construct
  source 1..15: mol_type = other DNA
tcatcaccgagcgtc

SEQ ID NO: 19 - DNA, 20 bp, synthetic construct
  source 1..20: mol_type = other DNA
ggtgatccacacagctgaca

SEQ ID NO: 20 - DNA, 23 bp, synthetic construct
  source 1..23: mol_type = other DNA
cctgttagttacctccccacatc

SEQ ID NO: 21 - DNA, 15 bp, synthetic construct
  source 1..15: mol_type = other DNA
ccaggaccatggtgc

SEQ ID NO: 22 - DNA, 15 bp, synthetic construct
  source 1..15: mol_type = other DNA
ccaggaccgtggtgc

SEQ ID NO: 23 - DNA, 20 bp, synthetic construct
  source 1..20: mol_type = other DNA
gctccagagaaggcatcact

SEQ ID NO: 24 - DNA, 22 bp, synthetic construct
  source 1..22: mol_type = other DNA
gccaagtctgaaccatgtgtca

SEQ ID NO: 25 - DNA, 15 bp, synthetic construct
  source 1..15: mol_type = other DNA
atacccgcacatgac

SEQ ID NO: 26 - DNA, 16 bp, synthetic construct
  source 1..16: mol_type = other DNA
catacccacacatgac

SEQ ID NO: 27 - DNA, 22 bp, synthetic construct
  source 1..22: mol_type = other DNA
gtccctagaaggccctagatgt

SEQ ID NO: 28 - DNA, 21 bp, synthetic construct
  source 1..21: mol_type = other DNA
gtgtggcacatttggtccatt

SEQ ID NO: 29 - DNA, 19 bp, synthetic construct
  source 1..19: mol_type = other DNA
ccaatcgccgagtcagagg

SEQ ID NO: 30 - DNA, 20 bp, synthetic construct
  source 1..20: mol_type = other DNA
cccagtccccagcactgtct

SEQ ID NO: 31 - DNA, 20 bp, synthetic construct
  source 1..20: mol_type = other DNA
aggggaggatagggtcagtg

SEQ ID NO: 32 - DNA, 21 bp, synthetic construct
  source 1..21: mol_type = other DNA
cctgttagttacctccccaca

SEQ ID NO: 33 - DNA, 20 bp, synthetic construct
  source 1..20: mol_type = other DNA
atacgtgcgtaagccaccac

SEQ ID NO: 34 - DNA, 20 bp, synthetic construct
  source 1..20: mol_type = other DNA
acccagatatgcccccttag

SEQ ID NO: 35 - DNA, 52 bp, synthetic construct
  source 1..52: mol_type = other DNA
ccggaattcgccgccaccatgggcagcacctgggggagccctggctgggtgc

SEQ ID NO: 36 - DNA, 32 bp, synthetic construct
  source 1..32: mol_type = other DNA
cgggcggccgctcagtgcctcttagccttgcc

SEQ ID NO: 37 - DNA, 52 bp, synthetic construct
  source 1..52: mol_type = other DNA
cgcggatccgccgccaccatggcggtgtctgccgggtccgcgcggacctcgc

SEQ ID NO: 38 - DNA, 47 bp, synthetic construct
  source 1..47: mol_type = other DNA
cgggcggccgctcagaactctgagtggacaggatcaggatttgactc

SEQ ID NO: 39 - protein, 5 aa, synthetic construct
FLEEL